(19) United States
(12) Patent Application Publication
(10) Pub. No.: US 2007/0122501 A1
(43) Pub. Date: May 31, 2007

(54) Formulations containing astragalus extracts and uses thereof

(75) Inventors: Calvin Bruce Harley, Murphys, CA (US); Allison C. Chin, Stanford, CA (US); Nancy Yuk-yu Ip, Kowloon (HK); Yung-huo Wong, Kowloon (HK); David M. Miller-Martini, Ridgway, PA (US)

Correspondence Address: PERKINS COIE LLP, P.O. BOX 2168, MENLO PARK, CA, 94026, US

(73) Assignees: Hong Kong University of Science and Technology, Kowloon (HK); Geron Corporation, Menlo Park, CA (US)

(21) Appl. No.: 10/563,533

(22) PCT Filed: Jun. 25, 2004
(86) PCT No.: PCT/US04/20363

§ 371 Date: Sep. 12, 2006

Related U.S. Application Data
(60) Provisional application No. 60/483,372, filed on Jun. 27, 2003.

(51) Int. Cl. A61K 36/48; A61K 36/28

(52) U.S. Cl. 424/757; 424/764

(57) ABSTRACT

Formulations containing plant extracts, in particular Astragalus extracts, and their use in inducing telomerase activity in cells, are described. Such compositions include pharmaceutical, including topical, and nutraceutical formulations. The methods and compositions are useful for treating diseases subject to treatment by an increase in telomerase activity in selected cells, such as, for example, HIV infection, various degenerative diseases, and acute or chronic skin ailments. They are also useful for enhancing replicative capacity of cells in culture, as in ex vivo cell therapy and proliferation of stem cells. Also described are cosmetic formulations of such extracts for conditioning the skin.

### FIELD OF THE INVENTION

The present invention relates to formulations containing plant extracts, and in particular to formulations containing Astragalus extracts, and their use in skin conditioning, in protecting the skin from UV damage, and in inducing telomerase activity in cells.

### BACKGROUND OF THE INVENTION AND REFERENCES

Astragalus Extracts

Herbal extracts of Astragalus species have been used as a traditional Chinese medicine. The extracted material is typically taken orally, as a nutritional supplement or a tea preparations. References relating to Astragalus species and extracts thereof include:
Abdallah, R. M. et al., “Astragalosides from Egyptian Astragalus spinosus Vahl”, Die Pharmazie (Germany) 48(6):4524 (June 1993).
Bedir, E. et al., “Cycloartane triterpene glycosides from the roots of Astragalus brachypterus and Astragalus microcephalus”, J. Natural Products 61(12): 1469-72 (December 1998).
Bedir, E. et al., “Trojanosides I-K: new cycloartane-type glycosides from the aerial parts of Astragalus trojanus”, Chem. Pharm. Bull. (Japan) 49(11):1482-6 (November 2001).
Ber, L. G., U.S. Pat. No. 5,786,343 (July 1998).
Calis, I. et al., “Cycloartane triterpene glycosides from the roots of Astragalus melanophrurius”, Planta medica (Germany) 63(2):183-6 (April 1997).
Calis, I. et al., “Four novel cycloartane glycosides from Astragalus oleifolius”, J. Natural Products 59(11):1019-23 (November 1996).
Calis, I. et al., “Secondary metabolites from the roots of Astragalus zahlbruckneri”, J. Natural Products 64(9):1179-82 (September 2001).
Chang, T. S., U.S. Pat. No. 5,942,233 (August 1999).
Chu, D. T. et al., “Fractionated extract of Astragalus membranaceus, a Chinese medicinal herb, potentiates LAK cell cytotoxicity generated by a low dose of recombinant interleukin-2”, Journal of clinical & laboratory immunology 26(4):183-7 (August 1988).
Chu, D. T. et al., “Immunotherapy with Chinese medicinal herbs. II. Reversal of cyclophosphamide-induced immune suppression by administration of fractionated Astragalus membranaceus in vivo”, Journal of clinical & laboratory immunology 25(3):125-9 (March 1988).
Dagarag, M. et al., “Differential impairment of lytic and cytokine functions in senescent human immunodeficiency virus type 1-specific cytotoxic T lymphocytes”, J Virol 77(5):3077-83 (March 2003).
Hasenoehrl, E. J. et al., U.S. Pat. No. 6,190,678 (February 2001).
Gariboldi, P. et al, “Cycloartane triterpene glycosides from Astragalus trigonus”, Phytochemistry 40(6):1755-60 (December 1995).
Huang, Y. et al., “Selected non-timber forest products with medicinal applications from Jilin Province in China”, Conference Title: Forest communities in the third millennium: Linking research, business, and policy toward a sustainable non-timber forest product sector; Kenora, Ontario, Canada, 1-4 Oct., 1999; General Technical Report-North Central Research Station, USDA Forest Service (No. NC-217): p. 93-101 (2000).
Pistelli, L., et al., “Antimicrobial and antifungal activity of crude extracts and isolated saponins from Astragalus verrucosus”, Fitoterapia 73(4):336-339 (2002).
Raman, A. et al, U.S. Pat. No. 6,346,539 (February 2002).
Semmar, N. et al., “Two new glycosides from Astragalus caprinus”, J. Natural Products 64(5):656-8 (May 2001).
Sinclair, S., “Chinese herbs: a clinical review of Astragalus, Ligusticum, and Schizandrae”, Alternative medicine review: a journal of clinical therapeutic 3(5):338-44 (October 1998).
Verotta, L. et al., “Cycloartane saponins from Astragalus peregrinus as modulators of lymphocyte proliferation”, Fitoterapia (Netherlands) 72(8): 894-905 (December 2001).
Watanabe, K. et al, “Cycloartane glycosides from the rhizomes of Cimicifuga racemosa and their cytotoxic activities”, Chem. Pharm. Bull. (Japan) 50(1): 121-5 (January 2002).
Zhang, Q. W. et al, “A new cycloartane saponin from Cimicifuga acerina”, Journal of Asian Nat. Prod. Res. 2(1):45-9 (1999).
Zhang, Q. W. et al, “Cycloartane glycosides from Cimicifuga dahurica”, Chem. Pharm. Bull. (Japan) 49(11):1468-70 (November 2001).
Zhao, K. S. et al, “Enhancement of the immune response in mice by Astragalus membranaceus extracts”, Immunopharmacology 20(3):225-33 (November-December 1990).
Zheng, Z. et al., “Studies on chemical constituents and immunological function activity of hairy root of Astragalus membranaceus”, Chinese journal of biotechnology 14(2):93-7 (1998).
Zhu, N. et al., “Cycloartane triterpene saponins from the roots of Cimicifuga foetida”, J. Natural Products 64 (5):627-9 (May 2001).
   Telomerase

Telomerase is a ribonucleoprotein that catalyzes the addition of telomeric repeats to the ends of telomeres. Telomeres are long stretches of repeated sequences that cap the ends of chromosomes and are believed to stabilize the chromosome. In humans, telomeres are typically 7-10 kb in length and comprise multiple repeats of the sequence TTAGGG. Telomerase is not expressed in most adult cells, and telomere length decreases with successive rounds of replication. After a certain number of rounds of replication, the progressive shortening of the telomeres results in the cells entering a telomeric crisis stage, which in turn leads to cellular senescence.

Certain diseases are associated with rapid telomeric loss, resulting in premature cell senescence. Expression of the gene encoding the human telomerase protein in human cells has been shown to confer an immortal phenotype, presumably though bypassing the cells' natural senescence pathway. In addition, expression of the telomerase gene in aging cells with short telomeres has been shown to produce an increase in telomere length and restore a phenotype typically associated with younger cells.

References discussing these and other characteristics of telomerase include:
Allsopp, R. C. et al., “Telomere shortening is associated with cell division in vitro and in vivo”, Exp. Cell Res. 220(1):194-200 (September 1995).
Bodnar, A. G. et al., “Extension of life-span by introduction of telomerase into normal human cells”, Science 279(5349):349-52 (Jan. 16, 1998).
Bodnar, A. G. et al., “Extension of life-span by introduction of telomerase into normal human cells” Science 279(5349):349-52 (Jan. 16, 1998).
Cech, T. et al., U.S. Pat. No. 6,261,836 (July 2001).
Chiu, C. P. et al., “Replicative senescence and cell immortality: the role of telomeres and telomerase” Proc. Soc. Exp. Biol. Med. 214(2):99-106 (February 1997).
Dagarag, M. et al., “Differential impairment of lytic and cytokine functions in senescent human immunodeficiency virus type 1-specific cytotox T lymphocytes”, J. Virol. 77(5):3077-83 (March 2003).
Farwell, D. G. et al., “Genetic and epigenetic changes in human epithelial cells immortalized by telomerase”, American Journal of Pathology 156(5):1537-47 (May 2000).
Fujimoto, R. et al., “Expression of telomerase components in oral keratinocytes and squamous cell carcinomas”, Oral Oncology 37(2):132-40 (February 2001).
Funk, Walter D. et al, “Telomerase expression restores dermal integrity to in vitro-aged fibroblasts in a reconstituted skin model”, JOURNAL: Experimental Cell Research 258(2):270-278 (Aug. 1, 2000).
Harle-Bachor, C. et al., “Telomerase activity in the regenerative basal layer of the epidermis inhuman skin and in immortal and carcinoma-derived skin keratinocytes”, Proc. Natl. Acad. Sci. USA 93(13):6476-81 (Jun. 25, 1996).
Harley, C. B. et al., “Telomerase and cancer”, Important Adv. Oncol. 57-67 (1996).
Harley, C. B. et al., “Telomerase, cell immortality, and cancer”, Cold Spring Harb. Symp. Quant. Biol. 59:307-15 (1994).
Harley, C. B. et al., “Telomeres and telomerase in aging and cancer”, Curr. Opin Genet. Dev. 5(2):249-55 (April 1995).
Harley, C. B. et al., “Telomeres shorten during ageing of human fibroblasts” Nature 345(6274):458-60 (May 31, 1990).
Harley, C. B., “Human ageing and telomeres”, Ciba Found. Symp. 211:129-39 (1997).
Harley, C. B., “Telomerase is not an oncogene”, Oncogene 21: 494-502 (2002).
Henderson, S. et al., “In situ analysis of changes in telomere size during replicative aging and cell transformation”, J. Cell Biol. 134(1):1-12 (July 1996).
Jiang, X. et al., PCT Pubn. No. WO 02/91999.
Jiang, X. R. et al., “Telomerase expression in human somatic cells does not induce changes associated with a transformed phenotype”, Nature Genetics 21(1): 111-4 (January 1999).
Kang, M. K. et al, “Replicative senescence of normal human oral keratinocytes is associated with the loss of telomerase activity without shortening of telomeres”, Cell Growth & Differentiation 9(1):85-95 (January 1998).
Kim, N. W. et al., U.S. Pat. No. 5,629,154 (May 1997).
Mattson, M. P., “Emerging neuroprotective strategies for Alzheimer's disease: dietary restriction, telomerase activation, and stem cell therapy”, Exp Gerontol. 35(4):489-502 (July 2000).
Morales, C. P. et al., “Absence of cancer-associated changes in human fibroblasts immortalized with telomerase”, Nature Genetics 21(1):115-8 (January 1999).
Oh, H. and Schneider, M. D., “The emerging role of telomerase in cardiac muscle cell growth and survival”, J Mol Cell Cardiol 34(7):717-24 (July 2002).
Simonsen, J. L. et al., “Telomerase expression extends the proliferative life-span and maintains the osteogenic potential of human bone marrow stromal cells”, Nat Biotechnol 20(6):592-6 (June 2002).
Villeponteau, B. et al., U.S. Pat. No. 5,583,016 (December 1996).
Yang, J. et al., “Human endothelial cell life extension by telomerase expression”, Journal of Biological Chemistry 274(37):26141-8 (Sep. 10, 1999).
Yang, J. et al, “Telomerized human microvasculature is functional in vivo”, Nature Biotechnology (United States) 19(3):219-24 (March 2001).
Yang, J., et al., “Human endothelial cell life extension by telomerase expression”, J. Biol. Chem. 274(37):26141-8 (Sep. 10, 1999).

### SUMMARY OF THE INVENTION

The invention described herein is generally related to the discovery of the ability of extracts of certain plant species, e.g. Astragalus or Cimicifuga species, to activate telomerase in cells. In particular embodiments, the plant is an Astragalus species, and in further embodiments the plant is Astragalus membranaceus. Aspects of the invention include formulations of such plant extracts for use in cosmetic, nutraceutical and pharmaceutical applications, in particular in applications where increasing telomerase activity in cells is desired.

Methods of using the plant extracts and formulations thereof for such applications are also provided, as are methods for formulating the plant extract, and methods of applying such formulations after the need for, or advantage of, increasing telomerase activity in cells or tissues has been determined.

The extract used in the formulation and methods of the invention is preferably obtained by extracting the plant material with a polar solvent selected from the group consisting of water, lower alcohols, lower alkyl esters, lower alkyl ketones, chloroform, and combinations thereof, preferably selected from water, lower alcohols, and combinations thereof. An exemplary solvent is one containing at least 75% ethanol and the remainder water, for example, 95% aqueous ethanol.

An extract of Astragalus membranaceus root may be obtained, for example, in an aqueous ethanolic solution, by a process that comprises:
(a) heating a mixture containing Astragalus membranaceus root in a solvent of 95% aqueous ethanol, preferably at reflux;
(b) filtering the solid from the resulting mixture;
(c) repeating steps (a)-(b) with the solid of step (b) and fresh solvent; and
(d) combining the filtrates from steps (b).
   The extract may also be obtained as a solid, by removing the solvent from the combined filtrates. In step (a) of the process, the mixture preferably includes about 0.1 g to 2.5 g of root per 5 ml of solvent, e.g. about 1 g/5 ml. Preferably, the mixture is kept at room temperature for about 0.5 to 1 hour prior to refluxing, and is refluxed for about 2 hours.

The concentration of the extract (based on weight of solid extract, exclusive of any solvent) in the formulation is preferably in the range of 0.1 to 100% (w/v). The extract may also be provided directly in solid form, without a vehicle or excipients. In this case, the extract is typically provided as a dry powder or in pressed form as a pill or tablet.

In further embodiments, the concentration is in the range of 0.1 to about 75% (w/v); e.g. 1 to 50, 5 to 25, or 10 to 15% (w/v). The extract is thus formulated in a vehicle that is appropriate for the relevant application, whether it be a cosmetic for topical use, nutraceutical for ingestion, or pharmaceutical for administration by a variety of routes including topical, oral or injection. By way of example, a cosmetic formulation will typically include the plant extract and one or more additional ingredients such as emulsifiers, thickeners, and skin emollients. In selected embodiments, the cosmetic formulation comprises an emulsifier and/or a skin emollient; in further embodiments, it comprises at least a skin emollient. The emulsifier, thickener, and/or skin emollient is one that is conventionally used in cosmetic formulations, as described further below.

In one aspect, the invention provides compositions and methods for conditioning the skin. Such methods comprise applying topically to the skin a formulation comprising an extract of an Astragalus or Cimicifuga species, preferably an extract of an Astragalus species, e.g. an Astragalus membranaceus extract, in a cosmetic formulation or vehicle.

In another aspect, the invention provides a method of increasing telomerase activity in a cell or tissue, comprising contacting the cell or tissue with a formulation comprising an extract of an Astragalus species, as described above. The biological activity of the subject extract is such that, when formulated in a solvent at a concentration of 25 μg/ml or less, it is effective to produce a level of telomerase activity in keratinocytes or fibroblasts, as measured in a TRAP assay, at least 50% greater than the level in said cells treated with said solvent, as measured in a TRAP assay as described herein. In further preferred embodiments, the extract is effective to produce a level of telomerase activity in keratinocytes or fibroblasts, as measured in a TRAP assay, at least 100% greater than the level in said cells treated with said solvent, as measured in a TRAP assay as described herein. The assay, which is described in detail below, comprises applying a composition to cells, typically keratinocytes or fibroblasts, and subsequently measuring telomerase activity in the cells.

Also described herein is a “scratch assay” in which the ability of a composition to increase the rate of closure of a scratch “wound” applied to a layer of cells, such as keratinocytes, is determined. The biological activity of the subject extract is preferably such that it is able to produce, at a concentration of 25 μg/ml or less, an amount of wound closure (wound healing activity) in a scratch assay of keratinocytes or fibroblasts, as described below, which is at least 25% greater than that seen in untreated or control cells. Even more potent activities may be appropriate for some applications, such as extracts that produce, at a concentration of 1 μg/ml or less, an amount of wound closure in a scratch assay of keratinocytes or fibroblasts which is at least about 50% or 100% greater than that seen in untreated or control cells.

The methods of the invention that are directed to enhancing telomerase activity in a cell or tissue, by contacting the cell or tissue with the extract or formulation, may further comprise a prior step of identifying a cell or tissue in which induction of telomerase is desired. This identification may entail, for example, diagnosing in a subject the presence of a condition associated with telomerase deficiency, or subject to treatment by increasing telomerase activity in cells or tissue of the subject. In a related aspect, the invention provides the use of a composition comprising an extract of an Astragalus or Cimicifuga species as described above, including the preferred embodiments described above, for the manufacture of a medicament for treating a condition subject to treatment by increasing telomerase activity in a cell or tissue.

The range of beneficial effects that may be achieved by telomerase activation include, for example: more rapid wound healing; the slowing of telomere loss occurring during aging of cells; postponing or reversing cellular senescence in disease conditions associated with cellular senescence; treating a disease condition associated with cells having a higher rate of cell division than normal cells of that cell type; treating a disease condition in which one or more cell types are limiting; and reducing telomere repeat loss while expanding cell number ex vivo, e.g. for use in cell-based therapies. Disease conditions subject to treatment by an increase in telomerase activity include HIV infection and degenerative disease, such as neurodegenerative disease, degenerative disease of the bones or joints, macular degeneration, atherosclerosis, and anemia, as discussed further below, as well as wounds or other acute or chronic condition of the epidermis. Also included are explant cells obtained from a patient, where the contacting is done ex vivo.

In a further aspect, the invention provides a cosmetic composition comprising an extract of an Astragalus species, as described above, present at a concentration (based on weight of solid extract, exclusive of solvent) of at least about 2.5% (w/v), up to about 75%, preferably up to about 50% (w/v), in a cosmetic formulation or vehicle. Preferably, the species is Astragalus membranaceus, and the extract is obtained from the root of the plant. The extract may be obtained by the processes described above and preferably has activity in a telomerase activation assay or wound closure assay as described above. The cosmetic formulation or vehicle will typically include at least one ingredient selected from the group consisting of an emulsifier, a thickener, and a skin emollient. The amount of the extract in the cosmetic composition is preferably at least about 5% (w/v), e.g. about 5 to 50% or 5 to 25% (w/v). In other embodiments the amount of the extract in the cosmetic composition may be at least about 10% (w/v), e.g. about 10 to 50% or about 10 to 25% (w/v).

In a further aspect, the invention provides a method of selecting an extract which is effective to increase telomerase activity in cells, by assaying a plant extract (preferably an extract of a flowering vascular plant, such as an herb) in a TRAP assay of keratinocytes or fibroblasts, as described further below, and selecting the extract if it produces a level of telomerase activity in the cells, at a concentration of 25 μg/ml, that is at least 50% greater than the level measured in a solvent control. The active extract can then be formulated in a pharmaceutical or cosmetic vehicle.

These and other objects and features of the invention will become more fully apparent when the following detailed description of the invention is read in conjunction with the accompanying drawings.

### BRIEF DESCRIPTION OF THE DRAWINGS

FIG. 1 shows the results of a TRAP assay of telomerase activity of an Astragalus membranaceus root extract, designated GRN139925, in young neonatal keratinocytes;

FIG. 2 shows the results of a similar assay in aging adult keratinocytes;

FIG. 3 shows the results of a similar assay in young neonatal fibroblasts;

FIGS. 4A-D and 5A-D show the results of screening Astragalus membranaceus root extracts, obtained using various solvents, for effectiveness in increasing telomerase activity;

FIG. 6 is a series of computer-generated images showing wound healing activity of an Astragalus membranaceus root extract, as measured in a “scratch assay” in aging adult keratinocytes;

FIG. 7 is a series of computer-generated images showing wound healing activity of an Astragalus membranaceus root extract, as measured in a “scratch assay” in late passage neonatal keratinocytes;

FIG. 8 is a series of computer-generated images showing wound healing activity of an Astragalus membranaceus root extract in aging adult keratinocytes, as compared to PDGF (platelet derived growth factor);

FIG. 9 is a graph showing the result of an assay such as shown in FIG. 7, run in the presence of mitomycin C, with data expressed as the percent of the “wound” area created by the scratch which remains (that is, is not covered by cells);

FIG. 10 is a graph showing the wound healing activity of an Astragalus membranaceus root extract in aging neonatal keratinocytes, in the presence or absence of a telomerase inhibitor, and in comparison to PDGF; and

FIGS. 11-12 show the results of short term (FIG. 11) and long term (FIG. 12) assays evaluating the protection of adult keratinocytes from cell death by an Astragalus membranaceus root extract and by AdhTERT (adenovirus human telomerase protein component).

### DETAILED DESCRIPTION OF THE INVENTION

I. Definitions

The following terms have the meanings given below, unless indicated otherwise:

“Conditioning the skin” includes such effects as improving the appearance, elasticity, thickness, or smoothness of skin, protecting skin from UV radiation, healing photodamaged skin, and/or reducing signs of skin aging, such as excessive dryness or wrinkling. Such signs of aging may be macroscopic, i.e. visually or tactilely apparent skin features, or they may be on a more microscopic or cellular level, such as cellular senescence of skin cells.

A “cosmetic vehicle” or “cosmetic formulation” or “cosmetically acceptable vehicle” refers to a composition formulated in a manner that is suitable for application to the skin, e.g. as a cream, gel, lotion, or ointment, and containing one or more components such as emulsifiers, surfactants, thickeners, emollients, and lubricants. Such a vehicle is not intended or necessarily suitable for internal consumption.

A “safe and effective amount” refers to an amount sufficient to induce a significant benefit, but to minimize undesired side effects; i.e. to provide a reasonable risk to benefit ratio.

By “effective to increase telomerase activity in a cell”, with reference to an extract as described herein, is meant that a composition containing the extract at a concentration of 25 μg/ml or less (based on solid weight) is effective to produce a level of telomerase activity in a keratinocyte or fibroblast cell, as measured in a TRAP assay as described herein, which is greater, by a factor of at least 1.5 (i.e. at least 50% greater), than the level produced by a similar formulation not containing the extract, as measured in a TRAP assay in said cells. In preferred embodiments, the extract is effective, at a concentration of 25 μg/ml or less, to produce a level of telomerase activity in such a cell, as measured in a TRAP assay as described herein, which is greater by a factor of at least 2 (i.e. at least 100% greater) than the level produced by a similar formulation not containing the extract.

In reference to administration of an extract to a patient, an “effective amount” refers to an amount of extract effective to increase telomerase activity in the cells or tissue of the patient, such that a desired therapeutic result is achieved. In reference to treatment of cells in vitro or ex vivo, an “effective amount” refers to an amount effective to increase telomerase activity in the cells, thereby increasing the replicative capacity and/or life span of the cells.

In concentrations expressed herein as % (w/v), 100% (w/v) corresponds to 1 g extract (solids)/ml vehicle. Concentrations present in formulations refer to weight of solid extract, exclusive of solvent.

A “vascular plant” refers to a plant which possesses a system of conducting tissue to transport water, mineral salts and sugars.

An “herb” refers to a plant whose stem does not produce woody, persistent tissue and generally dies back at the end of each growing season.

A plant “extract”, as used herein, refers to the material resulting from exposing a plant part to a solvent and removing the solvent. When the solvent used for extraction is retained rather than removed, an extract solution is obtained. The material may also undergo further purification, e.g. chromatographic separation into fractions.

“Lower alkyl” refers to an alkyl group having one to four carbon atoms, and, in selected embodiments, 1-2 carbon atoms (methyl or ethyl).

A “lower alcohol” is an alkanol having one to four carbon atoms; e.g., methanol, ethanol, isopropanol, or butanol. In selected embodiments, the lower alcohol is ethanol.

II. Telomerase Activating Extracts

A. Plant Sources

In one embodiment, the species extracted is Astragalus membranaceus, also referred to as Radix astragali, and also known by names such as Chinese Astragalus, Chinese milkvetch, Huang Qi, membranous milkvetch, and yellow vetch. However, extracts of other plant species, particularly other Astragalus species and species of Cimicifuga, having the desired properties may also be used. Indices of species of the Astragalus and Cimicifuga genus are available from various taxonomic reference sources, such as textbooks or online sources. For example, the Integrated Taxonomic Information System (IT IS) of the USDA maintains a searchable website, and a search of the genus Astragalus produced a list containing 619 “accepted” and 296 “not accepted” species. The website “GardenWeb” also includes a searchable database of various plant species, and a search of the genus Astragalus yielded information on 647 species. The genus Cimicifuga is much smaller, having about 15 species reported by these two sources.

Such species include, but are not limited to, species reported to contain components similar to components of A. membranaceus, such as A. trojanus (Bedir, 2001); A. zahlbruckneri (Calis, 2001); A. brachypterus, A. microcephalus (Bedir, 1998); A. peregrinus (Verotta, 2001); A. caprinus (Semmar, 2001); A. melanophrurius (Calis, 1997); A. oleifolius (Calis, 1996); A. trigonus (Gariboldi, 1995); A. spinosus (Abdallah, 1993); A. verrucosus (Pistelli, 2002); C. racemosa (Watanabe, 2002); C. dahurica (Zhang, 2001); C. foetida (Zhu, 2001); and C. acerina (Zhang, 1999), all cited above.

B. Extraction Methods

An exemplary procedure for extracting the plant material, which is not intended to be limiting, is as follows. In a preferred embodiment, the root of the plant is extracted; however, other plant parts, such as stems, leaves, or flowers, may also be employed.

A mixture containing the plant material, e.g. Astragalus membranaceus root segments, is heated in a polar solvent selected from the group consisting of water, lower alcohols, lower alkyl esters, lower alkyl ketones, chloroform, and combinations thereof. Preferably, the solvent is selected from the group consisting of water, lower alcohols, and combinations thereof. Exemplary solvents include 75% and 95% aqueous ethanol. The ratio of plant material to solvent is typically about 1-5 g or less for every 5 ml of solvent, more typically 1 g or less for every 5 ml of solvent. Heating the mixture generally comprises refluxing the solvent for about 0.5 to 3 hours, e.g. about 2 hours. The solid is then filtered from the mixture, and is preferably extracted again, in a similar manner, with fresh solvent. Again, the solid is filtered from the mixture, and the combined filtrates (solvent components) are concentrated to obtain the extract. In some cases, some or all of the extraction solvent may be retained, if its presence in the final formulation is acceptable.

An exemplary extraction procedure used to prepare an Astragalus membranaceus root extract, designated GRN925, is provided in Example 1.

III. Biological Activity

The extracts disclosed herein as active ingredients of the subject compositions are active in inducing telomerase activity in normal cells. Telomerase is a species-specific ribonucleoprotein that catalyzes the addition of oligonucleotide repeats (telomeres) onto the ends of chromosomal DNAS, to compensate for losses that occur with each round of DNA replication. Somatic cells, in contrast to tumor cells and certain stem cells, have little or no telomerase activity and stop dividing when the telomeric ends of at least some chromosomes have been shortened to a critical length, leading to programmed cellular senescence (cell death). Accordingly, an increase in telomerase activity is expected to inhibit cell death, and to promote cell proliferation.

A. TRAP Assay Protocol

The ability of a substance to increase telomerase activity in a cell can be determined using the TRAP (Telomeric Repeat Amplification Protocol) assay, which is known in the art (e.g. Kim et al., U.S. Pat. No. 5,629,154; Harley et al., U.S. Pat. No. 5,891,639). As used herein, “telomerase activity as measured in a TRAP assay” refers to telomerase activity as measured in keratinocytes or fibroblasts according to the following protocol. The activity is typically compared to the activity similarly measured in a control assay of such cells (e.g., a telomerase activity 50% greater than observed in a solvent control).

Cell lines suitable for use in the assay, preferably normal human fibroblasts (NHF) or normal human keratinocytes (NHK), can be obtained from commercial sources, such as Cascade Biologics, Portland, Oreg. or 4C Biotech, Seneffe, Belgium, or from the ATCC (American Type Culture Collection). ATCC normal human fibroblast cell lines, which can be located on the ATCC web site, include, for example, CCL135, CCL137, and CCL151.

Cells are plated at approx. 5000 cells/well, in growth medium (e.g. Epi-Life Medium+Keratinocyte Growth Factor Supplement+60 mM CaCl₂, supplied by Cascade Biologics, Inc.) for two days. Test compositions in a suitable solvent, such as 95% ethanol or DMSO, are added to selected wells in a range of concentrations and incubated for 16-24 hours. For the data reported herein, the solvent used was DMSO.

Cell lysing solution is prepared by addition of 3.0 mL Nonidet® P40, 1.0 mL CHAPS lysis buffer (see below), and 1.0 mL 10× TRAP buffer (see below) to 5.0 mL DNase-, RNase-free H₂O. (DNase-, RNase-free water may be generated by DEPC (diethylpyrocarbonate) treatment or purchased from vendors such as Sigma.)

The morphology of treated cells is first observed under a microscope, to verify that there are no visual signs of irregular growth. Media is removed from the wells, and the cells are rinsed twice in PBS (Ca and Mg free). The dishes are chilled, preferably on ice, and cell lysis buffer (see below) is added (approx. 100 μl per well) and triturated by pipetting up and down several times. The cells are the incubated on ice for 1 hour.

CHAPS Lysis Buffer

| | | |
|---|---|---|
| Stock | For 1 mL | Final concn. |

| 1 M Tris-HC1 pH 7.5 | 10 | μl | 10 | mM |
|---|---|---|---|---|
| 1 M MgCl₂ | 1 | μl | 1 | mM |
| 0.5 M EGTA | 2 | μl | 1 | mM |
| 100 mM AEBSF | 1 | μl | 0.1 | mM |
| 10% CHAPS^{a} | 50 | μl | 0.5% | |
| BSA | 1 | mg | 1 | mg/ml |
| 100% Glycerol | 100 | μl | 10% | |
| DNase-, RNase-free H₂O | 936 | μl (to 1 mL) | | |

^{a}The CHAPS detergent should be added just before use of the lysis buffer. In addition, AEBSF (4-(2-aminoethyl)-benzenesulfonyl fluoride hydrochloride) should be added to the lysis buffer just prior to performing the extraction step.

10× TRAP Buffer

| | | |
|---|---|---|
| Stock | For 5 ml | Final concn. |

| 1M Tris-HC1, pH 8.3 | 1 | ml | 200 | mM |
|---|---|---|---|---|
| 1M MgCl₂ | 75 | μl | 15 | mM |
| 1M KCl | 3.15 | ml | 630 | mM |
| Tween 20 (Boehringer Mannheim) | 25 | μl | 0.5% | |
| 0.1M EGTA | 500 | μl | 10 | mM |
| 20 mg/ml BSA | 250 | μl | 1 | mg/ml |

The following materials are combined to generate a master PCR Mix.

| Stock | Per Reaction (40 μl) | | Final concn.^{a} | |
|---|---|---|---|---|
| 10× TRAP Buffer | 5.0 | μl | 1× | |
| 2.5 mM dNTPs | 1.0 | μl | 50 | μM |
| Cy5-TS Primer (0.1 mg/ml) | 0.2 | μl | 0.4 | ng/ml |
| ACX Primer (0.1 mg/ml) | 1.0 | μl | 2 | ng/ml |
| TSU2 Int. Std. (1 pg/ml) | 1.0 | μl | 20 | fg/ml |
| U2 Primer (0.1 mg/ml) | 1.0 | μl | 2 | ng/ml |
| Taq Polymerase (5 U/μl) | 0.4 | μl | 2 | units |

| | |
|---|---|
| DNase-, RNase-free H₂O | 30.4 μl (to 40 μl total) |

^{a}Based on final volume of 40 μl PCR mix plus 10 μl cell lysate = 50 μl.

The PCR mix includes the following components: Cy5-TS primer, a 5′-Cy5 labeled oligonucleotide having the sequence 5′-AAT CCG TCG AGC AGA GTT-3′ (SEQ ID NO:1), is a telomerase substrate. Depending on the telomerase activity of the medium, telomer repeats (having the sequence . . . AGGGTT . . . ) will be added to the substrate, to form telomerase extended products, also referred to as telomerase products or TRAP products. The ACX primer, having the sequence 5′-GCG CGG CTT ACC CTT ACC CTT ACC CTA ACC-3′ (SEQ ID NO: 2), is an anchored return primer that hybridizes to the telomerase extended products.

The TSU2 internal standard, an oligonucleotide having the sequence 5′-AAT CCG TCG AGC AGA GTT AAA AGG CCG AGA AGC GAT-3′; SEQ ID NO:3), an extension of the TS primer sequence, is added in a small controlled quantity for quantitation purposes. The U2 primer, having the sequence 5′-ATC GCT TCT CGG CCT TTT (SEQ ID NO:4), is a return primer designed to hybridize to the 3′ region of the internal standard.

A sample of cell lysate (10 μL) is added to 40 μL of this PCR mix in a reaction tube, and the mixture is incubated at room temperature (30° C.) for 30 minutes. PCR is carried out by incubating the mixture at the following temperatures for the times indicated: 94° C./30 sec, 60° C./30 sec, and 72° C./30 sec; repeating this three-step cycle to conduct 20-30, preferably 31 cycles.

Loading dye containing e.g. bromophenol blue and xylene cyanol is added, and the samples are subjected to 10-15% non-denaturing PAGE in 0.6×TBE, until the bromophenol blue runs off the gel. Product formation is observed, e.g. by using a fluoroimager for detection of CY5-labeled telomerase products (maximal excitation at 650 nm; maximal emission at 670 nm).

The final amount of TSU2 internal standard after amplification is generally 5-10 amol per 50 μl reaction mixture. This internal control gives a specific 36-mer PCR amplification product that appears as a distinct band on the gel below the first telomer addition product (that is, the product of one telomer addition to the TS oligonucleotide, followed by amplification with the ACX return primer). This internal control band can be used to normalize the PCR amplifications from different samples.

The relative number of telomerase product molecules (TM) generated in the assay is determined according to the formula below:
TM=(T_{TRAP Product}−T_{BKD1})/(T_{Int Std}−T_{BKD2})
where: T_{TRAP Products} is the total intensity measured on the gel for all telomerase products, T_{BKD1} is the background intensity measured in a blank lane for an area equivalent in size to that encompassed by the telomerase products, Tint Std is the intensity for the internal standard band, and T_{BKD2} is the background intensity measured in a blank lane for an area equivalent in size to that encompassed by the internal standard band. The resulting number is the number of molecules of telomerase products generated for a given incubation time, which, for the purposes of determining TM, is designated herein as 30 minutes.

The activity of the subject extract is preferably such that, at a concentration of 25 μg/ml or less, it is able to produce a telomerase activity at least about 25% greater than that measured for a solvent control, and more preferably at least about 50% greater than that measured for a solvent control, as measured in a TRAP assay.

B. Exemplary TRAP Assay Results

Cell lines used in these experiments were obtained from Cascade Biologics, Inc. Neonatal keratinocytes (HEKneo-P) were obtained from a pool generated from three donors, designated HEKn(12), PD 8.46; HEKn(15), PD 9.3; and HEKn(16), PD 5.16. Adult keratinocytes were obtained from a single donor, designated HEKa(18). The terms “neonatal” and “adult” refer to the age of the donor, while the terms “young” and “aging” or “late passage” refer to the age of the cell culture, and correspond to the number of population doublings (indicated as PD no.).

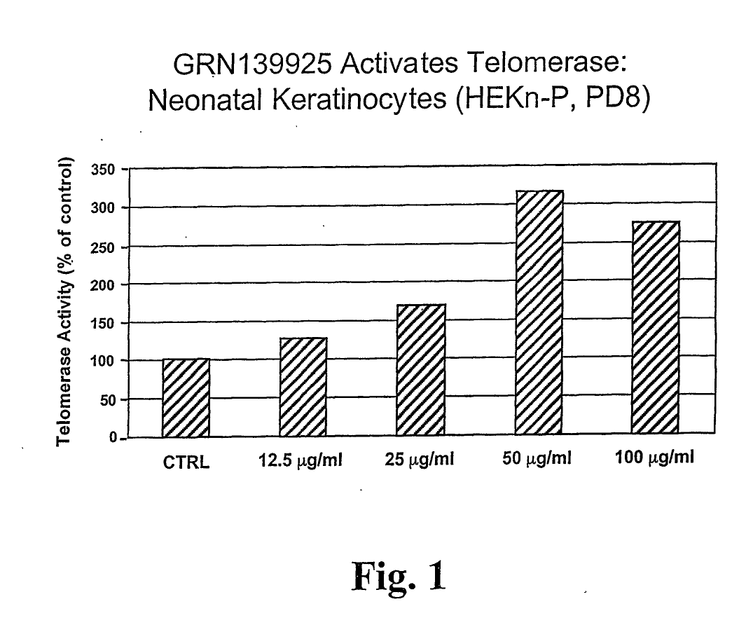

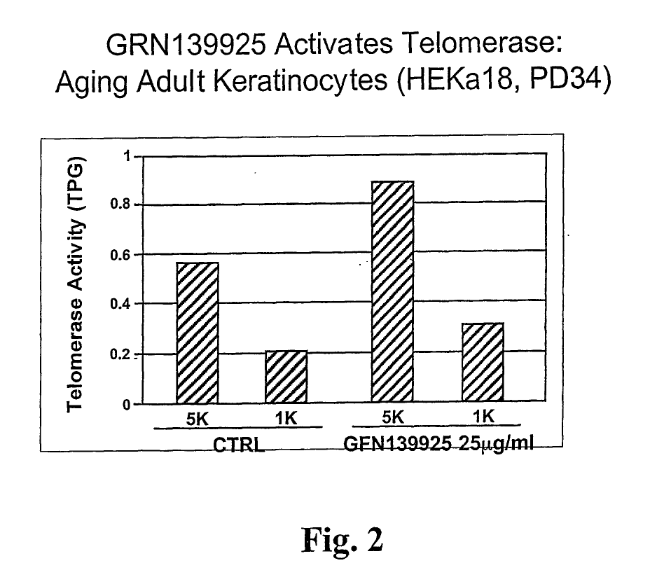

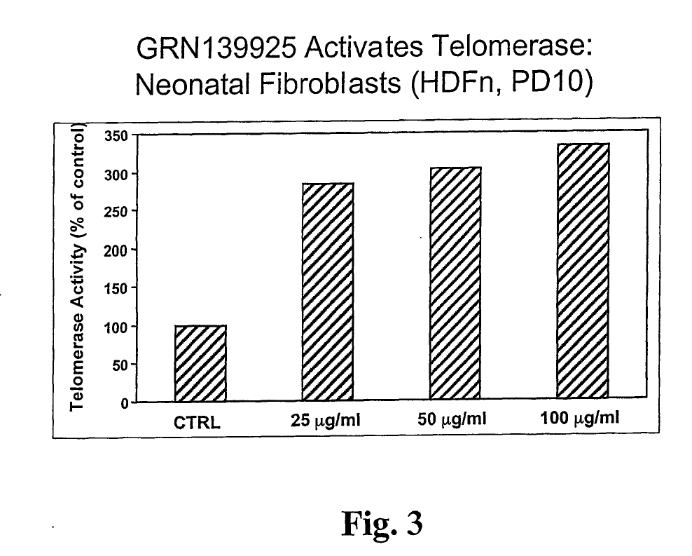

Activation of telomerase was evaluated for compositions containing a 95% aqueous ethanol extract of Astragalus membranaceus root, prepared according to the procedure described in Example 1, and designated herein as GRN925. Various formulations of the extract GRN925 were assayed for telomerase activation in young (PD8) neonatal keratinocytes (FIG. 1), aging (PD34) adult keratinocytes (FIG. 2) and young (PD10) neonatal fibroblasts (FIG. 3), using a TRAP (telomerase repeat amplification protocol) assay, as described above.

Data is presented in FIGS. 1 and 3 as telomerase activity relative to control, and in FIG. 2 as TPG, or “total product generated”, determined as described in U.S. Pat. No. 5,891,639. As can be seen from the Figures, telomerase activity was significantly greater than in the control in assays of compositions containing the extract at concentrations of 25 μg/ml or higher. For example, the composition of FIG. 1 containing 25 μg/ml of extract produced a telomerase activity at 170% of control.

The extract was shown not to increase telomerase activity in KB carcinoma cells, where such activity is already expected to be high (leading to abnormal proliferation). In this assay, telomerase activity was in fact lower in GRN925-treated cells (12 μg/ml and 25 μg/ml) than in the untreated cells.

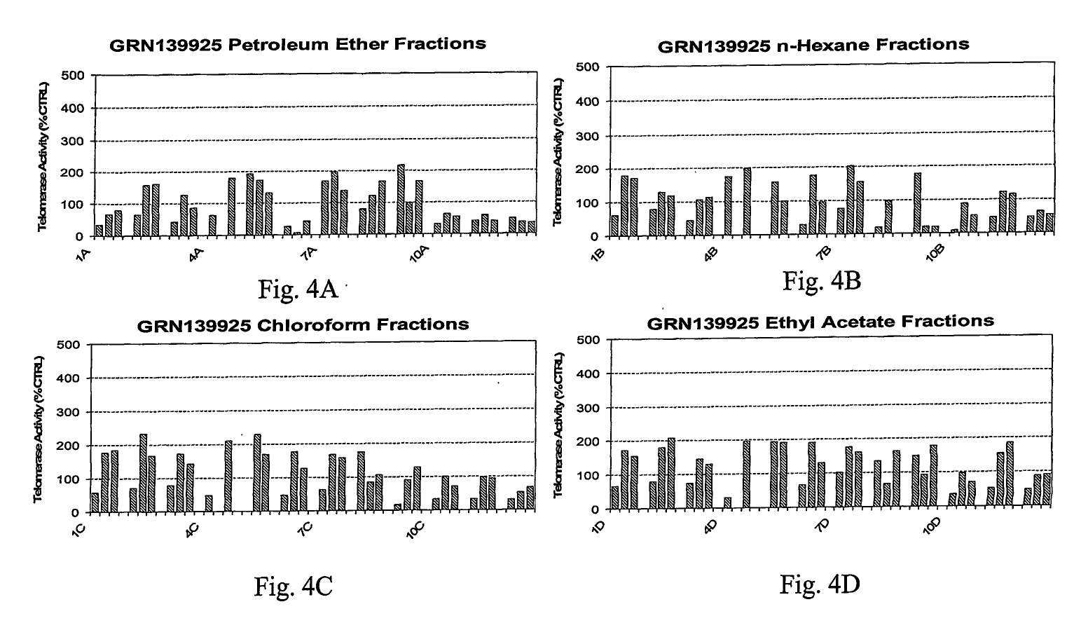

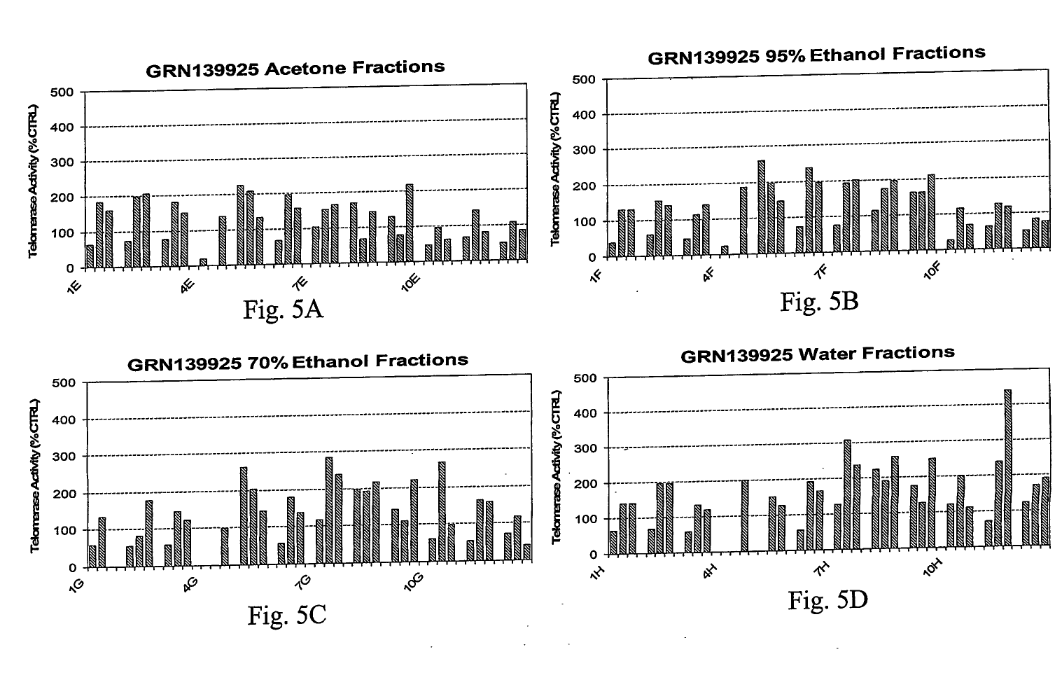

Screening assays were also conducted on compositions containing extracts obtained using several different solvents (pet ether, n-hexane, chloroform, ethyl acetate, acetone, 70% aqueous ethanol, 95% aqueous ethanol, and water) and fractionated by HPLC. As shown in FIGS. 4A-D and 5A-D, TRAP assays employing numerous fractions showed telomerase activity more than 100% greater than seen in control cells, with activity generally increasing in extracts from more polar solvents.

C. Wound Healing Assay Protocol

Wound healing activity of compositions containing the extracts can be evaluated, preferably in keratinocytes or fibroblasts, via a “scratch assay”, carried out as follows:

Cells are plated in flasks (5×10⁵ cells per flask) and cultured for two days in a humidified chamber at 5% CO₂, 37° C. To create the “wound”, a 2 ml plastic pipette is gently dragged to “scratch” the cell surface. The ideal wound is approximately 2-3 mm wide and 50 mm long (along the long axis of the tissue culture flask). The cells are retreated with medium containing either vehicle (DMSO; control sample) or test compositions at multiple concentrations. A wound area is identified, the flask marked, and the appearance of the cells documented photographically over 3-4 days continued culturing of the cells.

Amount of wound closure is determined by measuring the width of the wound over time for extract-treated samples relative to vehicle-treated or other control cells. Measurements are made from the photographs taken for each of the samples on days 1 (immediately after scratching), 2, 3, and 4. Percentage of wound healing is calculated by the following formula:
WH=100−[100×Wₙ/W₀],
where Wₙ is the width of the wound on day n and W₀ is the width of the wound on day one (immediately after scratching).

The activity of the subject extract is preferably such that it is able to produce, at a concentration of 25 μg/ml or less, an amount of wound closure (wound healing activity) in a scratch assay of keratinocytes or fibroblasts which is at least 25% greater than that seen in untreated or control cells. Even more potent activities may be appropriate for some applications, such as extracts that produce, at a concentration of 1 μg/ml or less, an amount of wound closure in a scratch assay of keratinocytes or fibroblasts which is at least about 50% or 100% greater than that seen in untreated or control cells.

D. Exemplary Scratch Assay Results

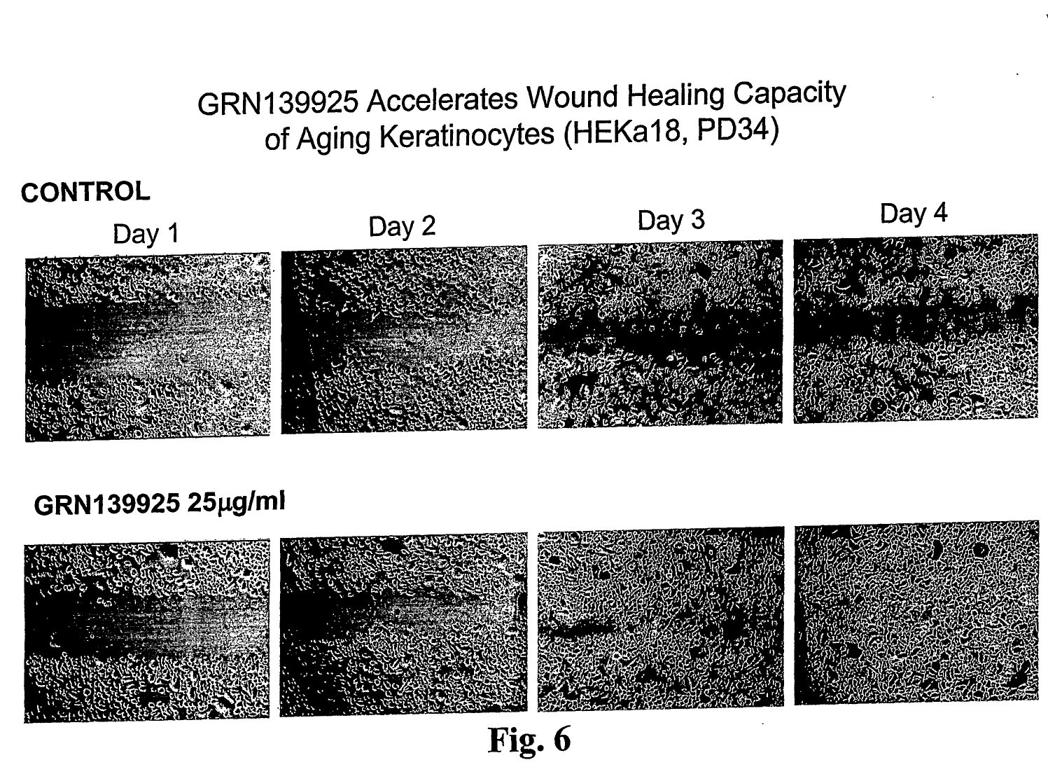

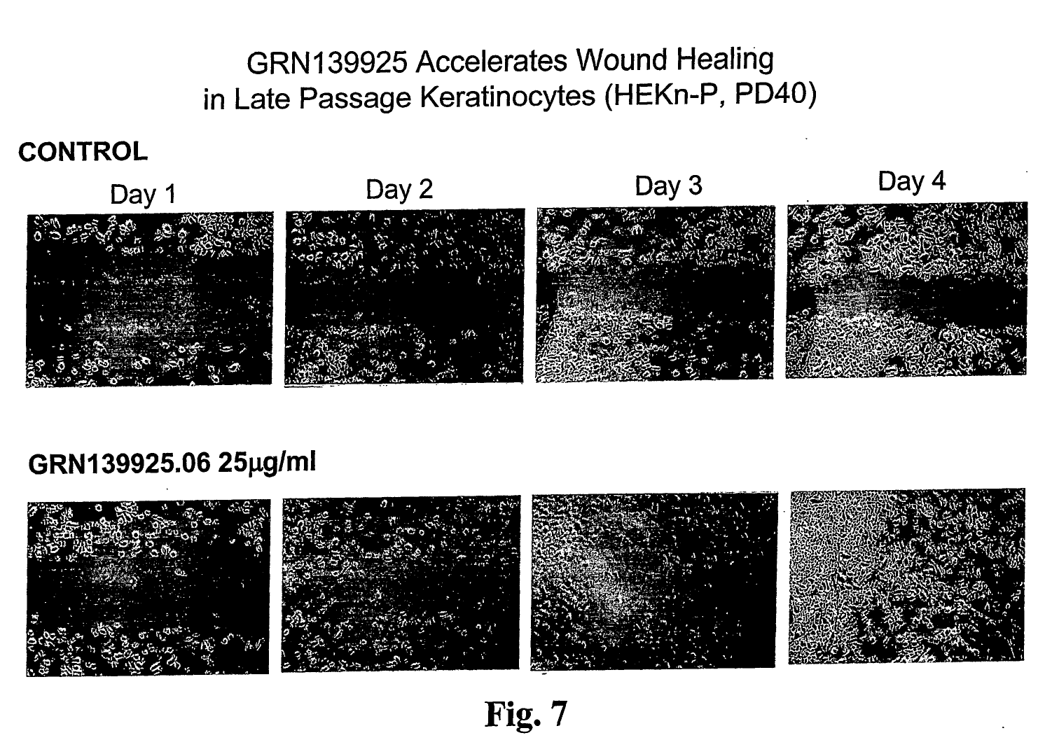

Results of typical scratch assays are shown in FIGS. 6-7, where the top row of images shows control cells, and the bottom row shows cells treated with 25 μg/ml GRN925. FIG. 6 shows an assay employing aging (PD34) adult keratinocytes, and FIG. 7 shows an assay employing late passage (PD40) neonatal keratinocytes. In both cases, the treated cells were nearly confluent at day 3 and fully confluent at day 4, in contrast to the control cells, in which a sizable “wound” remained at day 4.

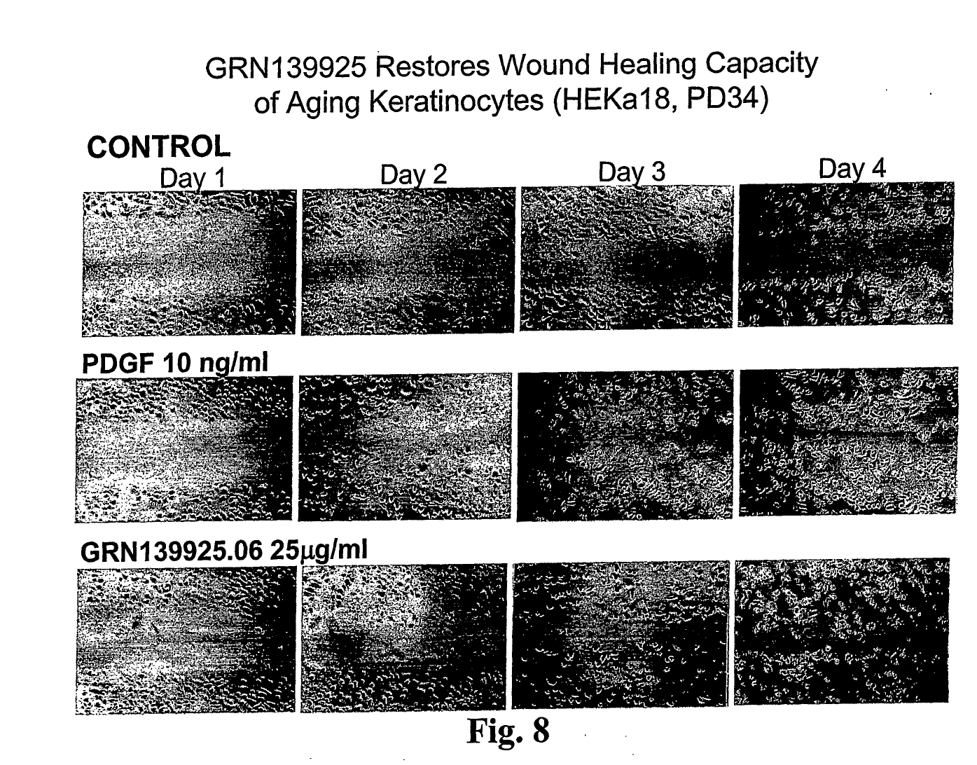

The activity of the extract in promoting healing was similar to that of PDGF (platelet derived growth factor), as shown in FIG. 8 (aging adult keratinocytes). Both treatments (second and third row of images) restored the cells to a confluent or near-confluent state by day 4, in contrast to the control cells (top row of images).

The following table shows WH_{cntrl} and WHₜₑₛₜ values for the compositions employed in the scratch assays shown in FIGS. 6-8, based on the results of those assays, using the formula shown above.

| Approx. wound width (arbitrary units) | | | | | |
|---|---|---|---|---|---|
| Day 1 | Day 4 | Day 1 | Day 4 | | |
| cntl | cntl | test | test | WHcntrl | WHtest |
| 18 | 8 | 14 | 0 | 55.6 | 100.0 |
| 22 | 9 | 19 | 0 | 59.1 | 100.0 |
| 22 | 11 | 17 | 4 | 50.0 | 76.5 |

A similar assay was run in the presence of 10 μM GRN163, a telomerase inhibitor. (GRN163 is an oligonucleotide that targets the template region of the telomerase RNA component. Specifically, GRN163 is a specific 3-mer N3′→P5′ thiophosphoramidate oligonucleotide; it is described in detail in PCT Pubn. No. WO 01/18015.) The results demonstrated that the wound healing activity of the extract composition is telomerase-dependent, as there was no apparent difference between the treated and the control cells in this assay.

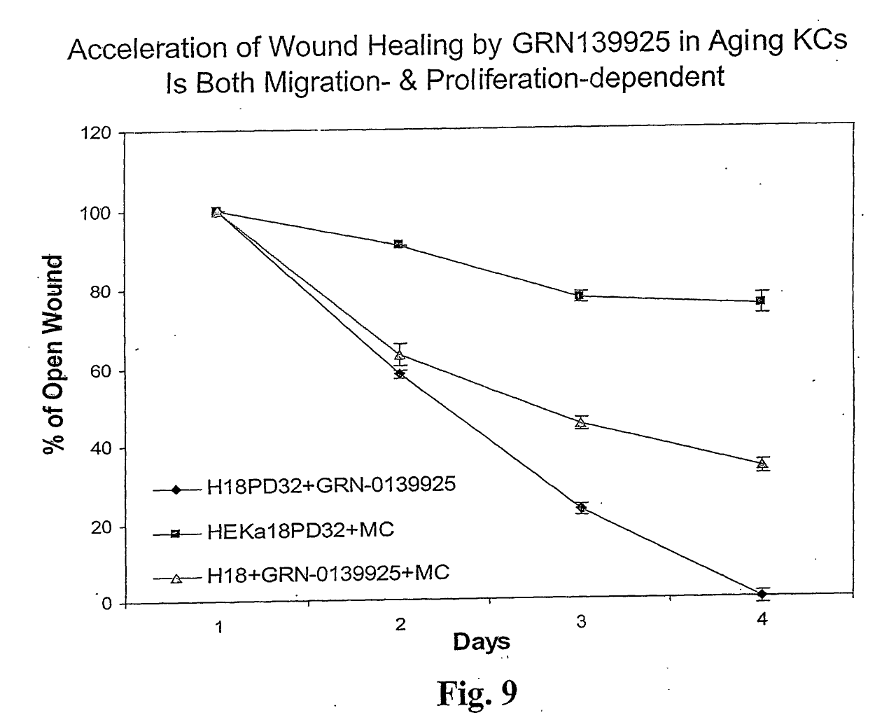

The wound healing activity of the extract composition was also reduced by the presence of an antiproliferative agent, mitomycin C, as shown in FIG. 9 (aging adult keratinocytes), demonstrating that the activity is both migration and proliferation dependent. A significant level of activity was retained under these conditions, providing evidence that telomerase activation can benefit non-dividing cells.

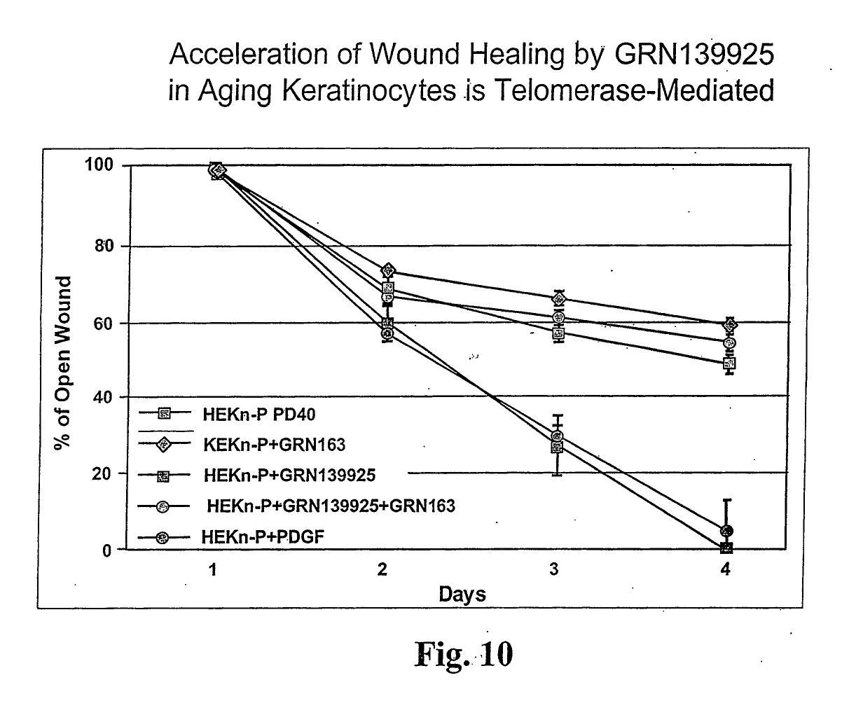

The wound healing activity of the extract composition, in the presence or absence of a telomerase inhibitor, and in comparison to PDGF (platelet derived growth factor), is shown graphically in FIG. 10 (aging neonatal keratinocytes). As shown, both the extract composition and PDGF produced complete or near-complete wound closure after four days, in contrast to control cells (about 50% closure) and cells treated with the extract and the telomerase inhibitor GRN163 (about 45% closure).

E. Inhibition of UV-Induced Cell Death

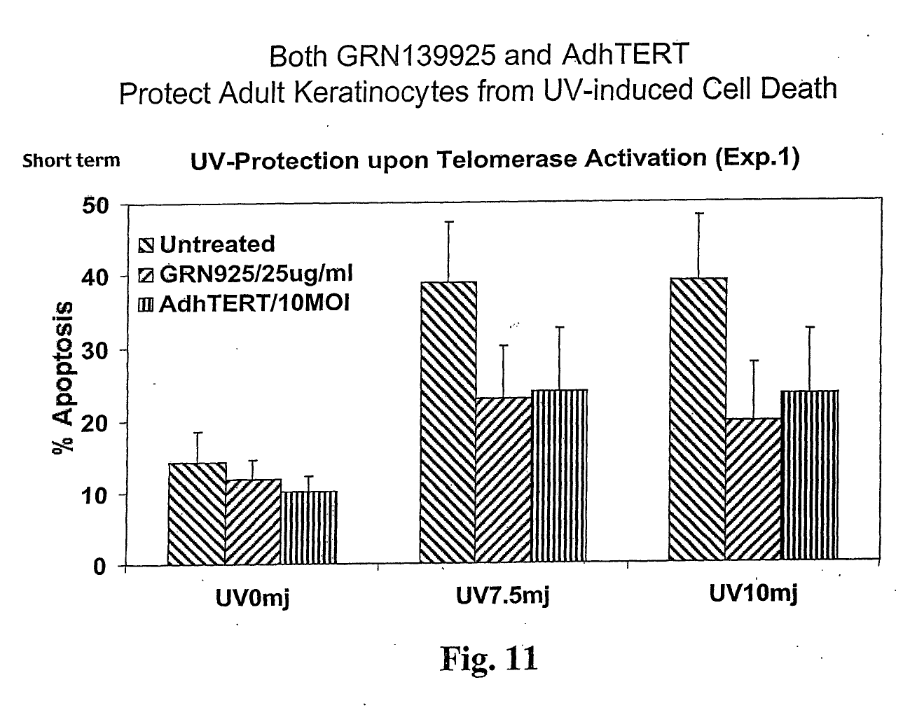

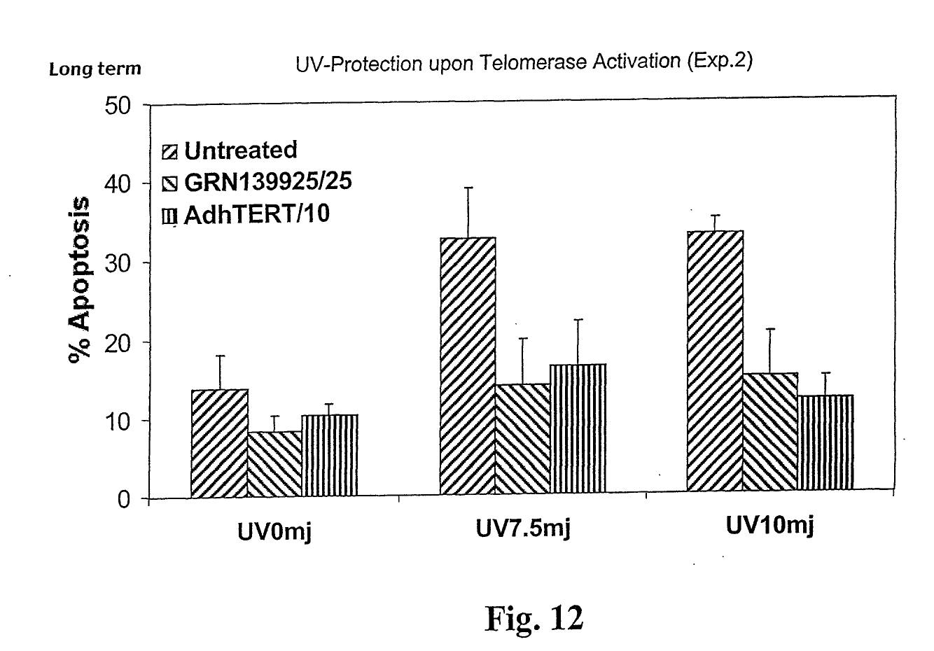

Protection of adult keratinocytes from cell death by the extract was comparable to that of AdhTERT (adenovirus human telomerase protein component), as shown in FIGS. 11-12. Both short term (FIG. 11) and long term (FIG. 12) photoaging was studied, according to the protocol described in Example 2.

As shown in FIGS. 11 and 12, minor differences in levels of apoptosis were observed between treated and untreated cells which were not exposed to UVB radiation. However, for cells exposed to 7.5-10 mJ/cm² UVB radiation, significant differences were observed, particularly in the long-term assay (FIG. 12). For example, in cells exposed to 7.5 mJ/cm² UVB, about 32% apoptosis was observed for untreated cells, but only about 13% apoptosis was observed for cells treated with 25 μg/ml GRN925.

IV. Cosmetic Compositions and Methods

In one aspect, the invention provides a method for conditioning the skin, regulating signs of skin aging, and/or protecting the skin from UV radiation. In accordance with the method, a formulation comprising, as an active ingredient, an extract of an Astragalus species, in a cosmetic vehicle, as defined further below, is applied topically to the skin. Also part of the invention is a cosmetic composition containing such an extract in a cosmetic vehicle. As demonstrated below, the composition is effective to induce telomerase activity in cells and to protect the cells from UV-induced apoptosis. Benefits of such activity are manifested in protection of skin from radiation, suppression of signs of aging in the skin, and promotion of healing.

The active ingredient (i.e. a telomerase inducing plant extract) can be present at various levels, i.e. about 0.1 to about 25% (w/v) or more in the cosmetic formulation, preferably up to about 75% (w/v), more preferably up to about 50% (w/v). In selected embodiments, the formulation contains about 1-10, 2.5-25, 5-10, 5-15, 5-25, 5-50, 10-15, 10-25, or 15-25% (w/v) of the extract. In other embodiments, the extract is present at least 5%, at least 10%, at least 15%, at least 20%, or at least 25% (w/v) of the formulation.

The formulation contains other ingredients conventional to cosmetic formulations, as described further below.

A. Cosmetic Vehicle

A1. Product Forms

The subject compositions may include a wide variety of product forms, including, for example, lotions, creams, gels, ointments, sticks, sprays, or pastes. These product forms may comprise several types of carriers, including, but not limited to, solutions, aerosols, emulsions, gels, solids, and liposomes. The carrier is frequently formulated as an emulsion, as described further below.

When the composition is formulated as an ointment, it may comprise a simple carrier base of animal or vegetable oils or semi-solid hydrocarbons, or an absorption ointment base which absorbs water to form an emulsion. Aerosols can be formed by adding a propellant, such as halogenated hydrocarbons known in the art, to a solution of the subject composition in a carrier such as described above. Aerosols are typically applied to the skin as a spray-on product.

The compositions of the present invention comprise a dermatologically acceptable carrier, within which the active ingredient and other components are incorporated, to allow these components to be delivered to the skin at an appropriate concentration. The carrier may contain one or more dermatologically acceptable solid, semi-solid or liquid fillers, diluents, solvents, extenders and the like. The carrier may be solid, semi-solid or liquid; preferred carriers are substantially liquid. The carrier should be physically and chemically compatible with the active ingredient and other components described herein. Preferred carriers contain a dermatologically acceptable, hydrophilic diluent; e.g., water, lower monovalent alcohols, low molecular weight glycols and polyols, such as propylene glycol, polyethylene glycol, polypropylene glycol, glycerol, butylene glycol, 1,2,4-butanetriol, sorbitol esters, 1,2,6-hexanetriol, and butanediol, ethoxylated ethers, propoxylated ethers, and combinations thereof. Water is a preferred diluent. The composition preferably comprises from about 60% to about 99% of the hydrophilic diluent.

In one embodiment, the formulation comprises an emulsion containing a hydrophilic phase, e.g., water or other hydrophilic diluent, and a hydrophobic phase, e.g., a lipid, oil or oily material, where one phase is dispersed in the other, continuous, phase. Examples are oil-in-water emulsions, water in oil emulsions, and water-in-silicone emulsions. Generally, the emulsion contains about 1% to 98% of the hydrophilic phase and about 1% to 50% of the hydrophobic phase. The emulsion may also comprise a gel network or a multiphase emulsion.

Preferred emulsions have an apparent viscosity at room temperature of from about 5,000 to about 200,000 centipoise (cps), depending on the physical form of the formulation. For example, a lotion may have an apparent viscosity of from about 10,000 to about 40,000 cps, and a cream may have an apparent viscosity of from about 60,000 to about 160,000 cps.

Suitable hydrophobic components employed in emulsions include, for example, vegetable oils, e.g. safflower oil, coconut oil, cottonseed oil, palm oil, soybean oil, and the like, which may be hydrogenated; animal fats and oils, such as lanolin; mineral oil; petrolatum, or petroleum jelly; or C7 to C40 hydrocarbons, e.g. such as dodecane, squalane, cholestanes, hydrogenated polyisobutylene, docosane, and various isoparaffins (branched hydrocarbons). Also suitable are esters of C1-C30 carboxylic acids and of C2-C30 dicarboxylic acids, where the alcohol component is derived from C1-C30 alcohols, glycols, or glycerols. Examples include, but are by no means limited to, isopropyl myristate, methyl palmitate, myristyl propionate, cetyl palmitate, dioctyl maleate, dioctyl sebacate, caprylic/capric triglyceride, PEG-8 caprylic/capric triglyceride, and ethylene glycol distearate; as well as propoxylated and ethoxylated derivatives thereof.

C1-C30 mono- and polyesters of sugars or other polyol moieties may also be used, as is known in the art, and include, for example, liquid materials such as glucose tetraoleate, glucose and mannose tetraesters of soybean oil fatty acids, galactose tetraesters of oleic acid, sorbitol hexaesters of unsaturated soybean oil fatty acids, and sucrose octaoleate. Solid materials include, for example, a sucrose polyester in which the degree of esterification is 7-8, and in which the fatty acid moieties are C18 mono- and/or di-unsaturated and behenic. Esters suitable for use in cosmetic emulsions are further described in, for example, U.S. Pat. Nos. 4,005,196, 5,306,516, 4,797,300, and 4,518,772. Also useful are C4-C20 alkyl ethers of polypropylene glycols, e.g. PPG-14 butyl ether, PPG-15 stearyl ether, dioctyl ether, dodecyl octyl ether, and mixtures thereof.

The hydrophobic component employed in an emulsion may also be an organopolysiloxane oil, such as disclosed in U.S. Pat. No. 5,069,897 (Orr). Examples of suitable organopolysiloxane oils include polyalkylsiloxanes, cyclic polyalkylsiloxanes, and polyalkylarylsiloxanes. Commercially available polyalkylsiloxanes include the polydimethylsiloxanes, which are also known as dimethicones, examples of which include the Vicasil™ series (General Electric) and the Dow Corning™ 200 series. Examples of alkyl-substituted dimethicones include cetyl dimethicone and lauryl dimethicone. Commercially available cyclic polyalkylsiloxanes include the cyclomethicones. Also useful are materials such as trimethylsiloxysilicate, such as that sold as a mixture with dimethicone as Dow Corning™ 593 fluid, and polyalkylaryl siloxanes.

A2. Components

Formulations of the present invention, particularly emulsions, preferably include one or more components selected from emulsifiers, surfactants, structuring agents, thickeners, and emollients, as described below.

A2(a). Emulsifiers and Surfactants

An emulsifier and/or surfactant is employed to disperse and suspend the discontinuous phase within the continuous phase. The surfactant should be hydrophilic enough to disperse in the hydrophilic phase; preferred surfactants are those having an HLB of at least about 8. The choice of surfactant will also depend upon the pH of the composition and the other components present.

Preferred hydrophilic surfactants are selected from nonionic surfactants, including those broadly defined as condensation products of long chain alcohols, e.g. C8-30 alcohols, with sugar or starch polymers, i.e., glycosides. Commercially available examples include decyl polyglucoside (available as APG 325 CS from Henkel) and lauryl polyglucoside (available as APG 600 CS and 625 CS from Henkel). Other useful nonionic surfactants include alkylene oxide esters and diesters of fatty acids, and alkylene oxide ethers of fatty alcohols, as well as the condensation products of alkylene oxides with both fatty acids and fatty alcohols. Nonlimiting examples of alkylene oxide-derived nonionic surfactants include ceteth-12, ceteareth-10, steareth-12, PEG-10 stearate, PEG-100 stearate, PEG-20 glyceryl stearate, PEG-80 glyceryl tallowate, PEG-30 glyceryl cocoate, PEG-200 glyceryl tallowate, PEG-8 dilaurate, PEG-10 distearate, and mixtures thereof. Still other useful nonionic surfactants include polyhydroxy fatty acid amides, such as coconut alkyl N-methyl glucoside amide.

The hydrophilic surfactants useful herein can also include any of a wide variety of cationic, anionic, zwitterionic, and amphoteric surfactants such as are known in the art. See, e.g., McCutcheon's, Detergents and Emulsifiers, North American Edition (1986), published by Allured Publishing Corporation; or U.S. Pat. No. 5,011,681 (Ciotti et al.). Cationic surfactants include, for example, cationic ammonium salts, such as quaternary ammonium salts, and amino-amides. Anionic surfactants include the alkoyl isethionates (e.g., C12-C30), alkyl and alkyl ether sulfates and phosphates, alkyl methyl taurates, and alkali metal salts of fatty acids. Examples of amphoteric and zwitterionic surfactants include derivatives of aliphatic secondary and tertiary amines in which one aliphatic substituent contains from about 8 to about 22 carbon atoms and one contains an anionic water solubilizing group, e.g., carboxy, sulfonate, sulfate, phosphate, or phosphonate. Examples are alkyl imino acetates, iminodialkanoates and aminoalkanoates, imidazolinium and ammonium derivatives. Other suitable amphoteric and zwitterionic surfactants include betaines, sultaines, hydroxysultaines, alkyl sarcosinates (e.g., C12-C30), and alkanoyl sarcosinates.

Silicone containing emulsifiers or surfactants include dimethicone copolyols, i.e. polydimethyl siloxanes having polyether side chains, as well as dimethicone copolyols modified with pendant alkyl, cationic, anionic, amphoteric, and zwitterionic moieties. Dimethicone copolyol emulsifiers useful herein are described, for example, in U.S. Pat. No. 4,960,764 (Figueroa, Jr. et al.); G. H. Dahms et al., Cosmetics & Toiletries, vol. 110, pp. 91-100, 1995; M. E. Carlotti et al., J. Dispersion Science & Technology, 13(3), 315-336 (1992); P. Hameyer, HAPPI 28(4), pp. 88-128 (1991); J. Smid-Korbar et al., Intl Journal of Cosmetic Science, 12, 135-139 (1990); and D. G. Krzysik et al., Drug and Cosmetic Industry, vol. 146(4) pp. 28-81 (1990).

A2(b). Structuring Agents

The subject formulations, particular when in the form of an oil-in-water emulsion, may contain a structuring agent, preferably at a level of about 2% to about 9%. Preferred structuring agents are those having an HLB (hydrophile-lipophile balance) of about 1-8 and a melting point of at least about 45° C. Suitable structuring agents include, for example, saturated C14 to C30 fatty alcohols or amines, which may contain 1 to about 5 moles of ethylene oxide; saturated C16 to C30 diols; saturated C16 to C30 monoglycerol ethers; C14 to C30 saturated fatty acids, which may be hydroxylated or ethoxylated; C14 to C30 saturated glyceryl monoesters having a monoglyceride content of about 40% or more; C14 to C30 saturated polyglycerol esters having from about 1 to about 3 alkyl groups and from about 2 to about 3 saturated glycerol units; C14 to C30 glyceryl monoethers; C14 to C30 sorbitan mono/diesters or saturated methyl glucoside esters, which may be ethoxylated and/or contain 1 to about 5 moles of ethylene oxide; C14 to C30 saturated sucrose mono/diesters; C14 to C30 saturated polyglucosides having an average of 1 to 2 glucose units, and mixtures thereof. In selected embodiments, the structuring agent includes stearyl alcohol, cetyl alcohol, behenyl alcohol, a polyethylene glycol ether of stearyl or cetyl alcohol having an average of about 2 ethylene oxide units, and mixtures thereof.

A2(c). Thickeners and Gelling Agents

The compositions of the present invention can also comprise a thickening or gelling agent, preferably in a level of about 0.1% to about 5%, more preferably from about 0.1% to about 3%, and most preferably from about 0.25% to about 2%. Nonlimiting classes of thickening agents include, for example, crosslinked polymers of acrylic acid, substituted acrylic acids, such as methacrylic acid, and salts and esters thereof, where the crosslinking agent is typically derived from a polyhydric alcohol. Preferred crosslinkers include allyl ethers of sucrose or of pentaerythritol. Also included are copolymers with acrylate esters, e.g. one short chain (C1-C4) and one long chain (C8-C40) acrylate ester. See, for example, U.S. Pat. No. 5,087,445 (Haffey et al.) or U.S. Pat. No. 4,509,949 (Huang et al.), as well as the CTFA International Cosmetic Ingredient Dictionary, fourth edition, 1991, pp. 12 and 80. Commercially available polymers of this type include the carbomers, which are homopolymers of acrylic acid crosslinked with allyl ethers of sucrose or pentaerytritol, e.g. the Carbopol™ 900 series (B.F. Goodrich), and acrylates/C10-30 alkyl acrylate crosspolymers, e.g. Carbopol™ 1342 and 1382 (B.F. Goodrich).

Crosslinked polyacrylate polymers are also used as thickeners or gelling agents, and include both cationic and nonionic polymers, with the cationic polymers being generally preferred. See, for example, U.S. Pat. No. 5,100,660 (Hawe et al.), U.S. Pat. No. 4,849,484 (Heard) U.S. Pat. No. 4,835,206 (Farrar et al.) U.S. Pat. No. 4,628,078 (Glover et al.), and U.S. Pat. No. 4,599,379 (Flesher et al.). In general, these materials are high molecular weight copolymers of a dialkylaminoalkyl acrylate monomer and a dialkylaminoalkyl methacrylate monomer, or their quaternary ammonium or acid addition salts, and may also incorporate any of a variety of unsaturated third monomers, such as ethylene, propylene, butylene, isobutylene, eicosene, maleic anhydride, acrylamide, methacrylamide, maleic acid, acrolein, cyclohexene, ethyl vinyl ether, and methyl vinyl ether. The alkyl portions of the acrylate monomers are preferably short chain alkyls such as C1-C8. Crosslinking agents, generally included to increase the viscoifying effect of the polymer, include methylenebisacrylamides, diallyldialkyl ammonium halides, polyalkenyl polyethers of polyhydric alcohols, allyl acrylates, vinyloxyalkylacrylates, and polyfunctional vinylidenes. Examples of such cationic polymers include Salcare™ SC92 and SC95 (Allied Colloids Ltd., Norfolk, Va.), both provided as mineral oil dispersions.

Also useful as thickeners are polymers such as high molecular weight polyacrylamides, including block copolymers of acrylamides and substituted acrylamides with acrylic acids and substituted acrylic acids, e.g. Hypan™ SR150H, SS500V, SS500W, and SSSA100H (Lipo Chemicals, Inc., Patterson, N.J.); polysaccharides, e.g. cellulose, carboxymethyl hydroxyethylcellulose, hydroxypropylcellulose, sodium cellulose sulfate, and the like, as well as alkyl substituted celluloses, such as cetyl hydroxyethylcellulose; crosslinked vinyl ether/maleic anhydride copolymers; and crosslinked poly(N-vinylpyrrolidones), as described, for example, in U.S. Pat. No. 5,139,770 (Shih et al.). Commercially available examples include ACP-1120, -1179, and -1180 (International Specialty Products, Wayne, N.J.).

Thickening and gelling agents may also include materials derived from natural sources, such as acacia, agar, algin, amylopectin, carrageenan, carnitine, dextrin, gelatin, gellan gum, guar gum, hectorite, hyaluroinic acid, hydrated silica, chitosan, kelp, locust bean gum, natto gum, tragacanth gum, xanthan gum, derivatives thereof, and mixtures thereof.

A2(d). Emollients

The subject formulations may also include a dermatologically acceptable emollient, e.g. at a level of about 2% to about 50%, depending on the physical form of the formulation. For example, lotions typically comprise about 5% to 10% emollient and about 60% to 80% water. A cream typically comprises about 10% to 20% emollient and about 50% to 75% water. An ointment may comprise about 2% to 10% emollient and about 0.1% to 2% of a thickening agent as described below. Generally, the emollient is present at a level of about 5% to about 25%.

Emollients are typically water-immiscible, oily or waxy materials which serve to lubricate the skin. An emollient may be selected from one or more of the following classes: triglyceride esters, which include, for example, vegetable and animal fats and oils; acetylated or ethoxylated glycerides; alkyl or alkyenyl esters of fatty acids, e.g. methyl palmitate, isopropyl isostearate, diisohexyl adipate, cetyl lactate, oleyl stearate, and the like; long chain fatty acids or alcohols such as myristic, palmitic, stearic, oleic, behenic, hydroxystearyl, and the like; lanolin and lanolin derivatives; polyhydric alcohol esters, e.g. mono and di-fatty acid esters of ethylene glycol, diethylene glycol, polyethylene glycol (200-6000), propylene glycol, and polypropylene glycol, and sorbitan, which may be ethoxylated; wax esters such as beeswax, spermaceti, and ethoxylated derivatives thereof; vegetable waxes such as carnauba and candelilla waxes; phospholipids such as lecithin and derivatives thereof, sterols, such as cholesterol and its fatty acid esters; and fatty acid amides.

Additional types of conditioning compounds include polyhydric alcohols and their derivatives, such as, for example, polypropylene glycol, hydroxypropyl sorbitol, pentaerythritol, xylitol, ethoxylated glycerol, soluble collagen, dibutyl phthalate, or gelatin. Also useful are ammonium and quaternary alkyl ammonium glycolates and lactates; aloe vera gel; and hyaluronic acid and derivatives thereof.

A2(e). Other Components

The formulations of the present invention may comprise a wide variety of additional components, as known in the art, including but not limited to anticaking agents, antimicrobial agents, astringents, opacifying agents, fragrances, pigments, preservatives, propellants, reducing agents, skin penetration enhancing agents, waxes, sunscreens, antioxidants and/or radical scavengers, chelating agents, sequestrants, anti-inflammatory agents, and vitamins. See, for example, Harry's Cosmeticology, 7th Ed., Harry & Wilkinson (Hill Publishers, London 1982); Pharmaceutical Dosage Forms-Disperse Systems; Lieberman, Rieger & Banker, Vols. 1 (1988) & 2 (1989); Marcel Decker, Inc.; The Chemistry and Manufacture of Cosmetics, 2nd. Ed., deNavarre (Van Nostrand 1962-1965); and The Handbook of Cosmetic Science and Technology, 1st Ed. Knowlton & Pearce (Elsevier 1993).

Such additional components should be physically and chemically compatible with the vehicle and active components described herein, and not unduly impair stability, efficacy or other use benefits associated with the compositions of the present invention.

The compositions of the present invention are preferably formulated to have a pH of 10.5 or below, more preferably from about 3-8, and most preferably from about 5-8.

B. Methods of Application

A formulation of a biologically active extract as described above, in a cosmetic vehicle such as described above, is applied in an amount and frequency effective to obtain the desired effects, which include, for example, diminishing roughness and dryness of the skin, diminishing or preventing wrinkling, and preventing or alleviating damage of skin by UV radiation. The formulation is also beneficial in healing of superficial wounds or abrasions of the skin.

Quantities of the present compositions applied per application can vary, but are typically from about 0.1 to about 10 mg/cm² of skin surface. In one embodiment, the formulation is applied daily, to areas such as the face, hands, arms, or feet; however, application rates can vary from about once per week up to about three times per day or more. The compositions may also be applied, generally in the form of a skin lotion or cream, to be left on the skin for an extended period such as one or more hours, for some aesthetic, prophylactic, therapeutic or other benefit (i.e., a “leave-on” composition).

V. Therapeutic Compositions and Methods

A. Formulations and Methods of Administration

Pharmaceutical formulations containing the telomerase activating extracts described herein may take the form of solid, semi-solid, lyophilized powder, or liquid dosage forms, such as, for example, tablets, capsules, powders, sustained-release formulations, solutions, suspensions, emulsions, suppositories, creams, ointments, lotions, aerosols, or the like, preferably in unit dosage forms suitable for simple administration of precise dosages.

The compositions typically include a conventional pharmaceutical carrier or excipient and may additionally include other medicinal agents, carriers, adjuvants, and the like. Typically, the composition will contain about 0.1% to 100% (w/v), preferably 0.5% to 50% (w/v), more preferably 2.5 to 25% (w/v), of a telomerase inducing extract as described herein, with the remainder consisting of suitable pharmaceutical excipients. As noted above, the extract may also be provided without a vehicle or excipients, e.g. as a dry powder or in pressed pill or tablet form, for oral administration.

Further exemplary concentrations of the extract in a formulation are in the range of 0.5 to 75, 1 to 50, 5 to 25, 10 to 25, 10 to 50, 20 to 75, 20 to 50, and 20 to 25% (w/v). Choice of a suitable concentration depends on factors such as the desired dose, frequency and method of delivery of the active agent.

For oral administration in solid form, or for preparation of a nutraceutical formulation, suitable excipients, when present, can include pharmaceutical grades of carriers such as mannitol, lactose, glucose, sucrose, starch, cellulose, gelatin, magnesium stearate, sodium saccharine, and/or magnesium carbonate. For use in oral liquid preparation, the composition may be prepared as a solution, suspension, emulsion, or syrup, being supplied either in solid or liquid form suitable for hydration in an aqueous carrier, such as, for example, aqueous saline, aqueous dextrose, glycerol, or ethanol, preferably water or normal saline. If desired, the composition may also contain minor amounts of non-toxic auxiliary substances such as wetting agents, emulsifying agents, or buffers.

The composition may be administered to a subject by a variety of known routes, e.g. orally or parenterally; that is, by intravenous, intrathecal, subcutaneous, intraperitoneal, or intramuscular injection; by inhalation, topically, or transdermally.

For parenteral administration, an injectable composition typically contains a formulation of a telomerase activating extract in a suitable IV solution, such as sterile physiological salt solution. The composition may also formulated as a suspension in a lipid or phospholipid, in a liposomal suspension, or in an aqueous emulsion.

The extract compositions may also be delivered transdermally, typically by the use of a transdermal patch which allows for continuous slow delivery to a selected skin region. It may be desirable to include permeation enhancing substances, such as fat soluble substances (e.g., aliphatic carboxylic acids, aliphatic alcohols), or water soluble substances (e.g., alkane polyols such as ethylene glycol, 1,3-propanediol, glycerol, propylene glycol, and the like).

Methods for preparing such dosage forms are known or will be apparent to those skilled in the art; for example, see Remington's Pharmaceutical Sciences (19th Ed., Williams & Wilkins, 1995). The composition to be administered will contain a quantity of the extract in a pharmaceutically safe and effective amount for effecting telomerase induction in the target cells or tissue.

For therapeutic treatment of a subject or patient, such as a mammal or a human patient, dosage levels of telomerase activating extract are determined based on factors such as the weight and overall health of the subject, the condition treated, severity of symptoms, etc., but are typically in the range of about 1 to 50 g/day or more for a human patient, typically about 4 to 40 g/day.

For administration as a nutraceutical, e.g. a dietary supplement, dosages are typically in the range of 20 mg-1 g/day, typically 20-200 mg/day, for a human subject.

B. Therapeutic Indications

The present invention provides methods for increasing telomerase activity in a cell, by contacting a cell or tissue with an extract as described herein, in an amount effective to increase telomerase activity in the cell. The method may include the preliminary step of identifying a cell or tissue in which an increase in telomerase activity is desired. The cell may be in culture, i.e. in vitro or ex vivo, or within a subject or patient in vivo.

Benefits to be realized from an increase in telomerase activity in a cell or tissue include, for example, enhancement of the replicative capacity and/or life span of the contacted cells. The method may further comprise diagnosing a condition in a subject or patient wherein an increase in telomerase activity in cells or tissue of the patient is desired; e.g., diagnosing a disease subject to treatment by an increase in telomerase activity in cells or tissue. Accordingly, the invention provides methods of treating a condition in a patient, by increasing telomerase activity in cells or tissue of said patient, the method comprising administering to a subject in need of such treatment an effective amount of an extract as described herein. An “effective amount” refers to an amount effective to increase telomerase activity in the cells or tissue of the patient, such that a therapeutic result is achieved.

Such conditions may include, for example, conditions associated with cellular senescence or with an increased rate of proliferation of a cell in the absence of telomerase, which leads to accelerated telomere repeat loss. By “increased rate of proliferation” is meant a higher rate of cell division compared to normal cells of that cell type, or compared to normal cells within other individuals of that cell type. The senescence of those groups of cells at an abnormally early age can eventually lead to disease (see West et al., U.S. Pat. No. 6,007,989).

Various disease states exist in which an increase in telomerase activity in certain cell types can be beneficial. Accordingly, the invention provides methods of treating in a patient a condition selected from the following, by increasing telomerase activity in the cells of the patient, comprising administering to a subject in need of such treatment, an effective amount of an extract as described herein. In some cases, the condition may also be subject to treatment by ex vivo cell therapy, as described further below, employing the associated cell types (indicated in parenthesis).

(a) Alzheimer's disease, Parkinson's disease, Huntington's disease, and stroke (cells of the central nervous system, including neurons, glial cells, e.g. astrocytes, endothelial cells, fibroblasts),

(b) age-related diseases of the skin, such as dermal atrophy and thinning, elastolysis and skin wrinkling, sebaceous gland hyperplasia or hypoplasia, senile lentigo and other pigmentation abnormalities, graying of hair and hair loss or thinning, or chronic skin ulcers (fibroblasts, sebaceous gland cells, melanocytes, keratinocytes, Langerhan's cells, microvascular endothelial cells, hair follicle cells),

(c) degenerative joint disease (cells of the articular cartilage, such as chondrocytes and lacunal and synovial fibroblasts),

(d) osteoporosis and other degenerative conditions of the skeletal system (cells of the skeletal system, such as osteoblasts, bone marrow stromal or mesenchymal cells, osteoprogenitor cells),

(e) age- and stress-related diseases of the vascular system including atherosclerosis, calcification, thrombosis, and aneurysms (cells of the heart and vascular system, including endothelial cells, smooth muscle cells, and adventitial fibroblasts),

(f) age-related macular degeneration (cells of the eye, such as pigmented epithelium and vascular endothelial cells),

(g) AIDS (HIV-restricted CD8⁺ cells); and

(h) age- and stress-related immune system impairment, including impairment of tissue turnover, which occurs with natural aging, cancer, cancer therapy, acute or chronic infections, or with genetic disorders causing accelerated cell turnover, and related anemias and other degenerative conditions (other cells of the immune system, including cells in the lymphoid, myeloid, and erythroid lineages, such as B and T lymphocytes, monocytes, circulating and specialized tissue macrophages, neutrophils, eosinophils, basophils, NK cells, and their respective progenitors).

In addition to the cell types noted above, further cell types in which an increase in telomerase activity can be therapeutically beneficial include, but are not limited to, cells of the liver, endocrine and exocrine glands, smooth musculature, or skeletal musculature.

As an example, in the case of HIV-infected individuals, CD8⁺ cell turnover is increased as these cells attempt to control the level of HIV-infected CD4⁺ cells. In AIDS (item (g) above), disease is believed to be caused by the early senescence of HIV-restricted CD8⁺ cells. The aging of such cells is attributed not simply to abnormal amount of loss of telomere sequences per cell doubling, but, in addition, to the increased replicative rate of the cells, such that telomere attrition is greater than normal for that group of cells. The invention thus provides methods of treating an HIV infected subject, and more particularly of reducing early senescence of HIV-restricted CD8⁺ cells in an HIV infected subject, by administering to a subject in need of such treatment an effective amount of an extract as described herein.

An increase in telomerase activity can benefit non-dividing cells as well as proliferating cells, e.g. in conditions associated with increased susceptibility to cell death due to stress, such as ischemia in heart failure or in stroke (see e.g. Oh and Schneider, J Mol Cell Cardiol 34(7):717-24; Mattson, Exp Gerontol. 35(4):489-502). The invention thus provides methods of reducing stress- or DNA-damage-induced cell death in a subject, such as a subject experiencing ischemic conditions in tissue due to heart failure or stroke, by increasing telomerase activity in cells of the subject, comprising administering to a subject in need of such treatment an effective amount of an extract as described herein. As noted above, the method may include the preliminary step of diagnosing in the subject the indicated condition.

In another aspect, the compositions may be used for the treatment of individuals in which one or more cell types are limiting in that patient, and whose life can be extended by extending the ability of those cells to continue replication or resist stress-induced cell death. One example of such a group of cells is lymphocytes present in Down's Syndrome patients. The invention thus provides a method of enhancing replicative capacity and/or life span of lymphocytes present in a Down's Syndrome patient, by increasing telomerase activity in said cells of the patient, comprising administering to such a patient an effective amount of an extract as described herein. The compositions may also be used to improve resistance to stress-induced cell death occurring during normal aging.

In a further aspect of the invention, increasing telomerase activity is effective to promote healing of wounds, burns, abrasions or other acute or chronic conditions of the epidermis. The invention thus provides a method of treating an acute or chronic condition of the epidermis, by administering to a patient in need of such treatment, preferably topically to the affected area, an effective amount of an extract as described herein.

As used herein, an “acute or chronic condition of the epidermis” includes acute conditions such as lesions suffered in trauma, burns, abrasions, surgical incisions, donor graft sites, and lesions caused by infectious agents, and chronic conditions such as chronic venous ulcer, diabetic ulcer, compression ulcer, pressure sores, and ulcers or sores of the mucosal surface. Also included are skin or epithelial surface lesions caused by a persistent inflammatory condition or infection, or by a genetic defect (such as keloid formation and coagulation abnormalities). See, for example, PCT Pubn. No. WO 02/91999.

Desirable effects of an increase in telomerase activity in such treatment include cell proliferation or migration at the treatment site, epithelialization of the surface, closure of a wound if present, or restoration of normal physiological function. By “epithelialization” or “reepithelialization” of a treatment site is meant an increase in density of epithelial cells at the site as a result of the applied therapy.

The method may also be used to enhance growth of engrafted cells. Desirable effects of an increase in telomerase activity in such treatment include coverage of the treatment site, survival of engrafted cells, lack of immune rejection, closure of a wound if present, or restoration of normal physiological function. Engrafted cells may participate in wound closure either by participating directly in the healing process (for example, becoming part of the healed tissue), or by covering the wound and thereby providing an environment that promotes healing by host cells.

The invention also contemplates manipulation of the skin and repair of any perceived defects in the skin surface for other purposes, such as cosmetic enhancement.

In a further aspect, the methods and compositions of the invention can be used to enhance replicative capacity and/or extend life span of cells in culture, e.g. in ex vivo cell therapy or in monoclonal antibody production, by increasing telomerase activity in the cells. Increasing telomerase activity increases the replicative capacity of such cells by slowing telomere repeat loss and/or improving resistance to stress-induced cell death during cell proliferation.

In the case of ex vivo applications, an effective amount of an extract as described herein is added to explant cells obtained from a subject. An “effective amount” refers to an amount effective to increase telomerase activity in the cells, thereby increasing the replicative capacity and/or life span of the cells.

The explant cells may include, for example, stem cells, such as bone marrow stem cells (U.S. Pat. No. 6,007,989), bone marrow stromal cells (Simonsen et al., Nat Biotechnol 20(6):592-6, 2002), or adrenocortical cells (Thomas et al, Nat Biotechnol 18(1):39-42, 2000). Disease conditions such as those noted in items (a)-(h) above may also be subject to ex vivo cell-based therapy. Examples include the use of muscle satellite cells for treatment of muscular dystrophy, osteoblasts to treat osteoporosis, retinal pigmented epithelial cells for age-related macular-degeneration, chondrocytes for osteoarthritis, and so on.

For example, the recognition that functional CD8⁺ cells are limiting in AIDS patients to control the expansion of infected CD4⁺ cells allows a therapeutic protocol to be devised in which HIV-restricted CD8⁺ cells are removed from an HIV-infected individual at an early stage, when AIDS is first detected, stored in a bank, and then reintroduced into the individual at a later stage, when that individual no longer has the required CD8⁺ cells available. Thus, an individual's life can be extended by a protocol involving continued administration of that individual's limiting cells at appropriate time points. These appropriate points can be determined by following CD8⁺ cell senescence, or by determining the length of telomeres within such CD8⁺ cells, as an indication of when those cells will become senescent. In accordance with the invention, the stored cells can be expanded in number in the presence of an agent which slows telomere repeat loss, i.e. an extract as described herein.

Accordingly, the invention provides methods of ex vivo cell based therapy, which include obtaining a cell population from a subject, and expanding the cell population ex vivo, wherein the cell population is treated with an extract as described herein, in an amount effective to increase telomerase activity and thereby enhance the replicative capacity and/or life span of the cell population. The method generally includes diagnosing in a subject a condition subject to treatment by ex vivo cell based therapy, such as those noted above.

In a further embodiment, the invention provides a method of stem cell proliferation, wherein a stem cell population is treated with an extract as described herein, in an amount effective to increase telomerase activity and thereby enhance the replicative capacity and/or life span of the cell population.

VI. Selection of Additional Extracts Effective to Increase Telomerase Activity

The invention also provides methods of selecting a plant extract which is effective to increase telomerase activity in cells, by screening plant extracts in accordance with the TRAP assay described in Section III, and selecting an extract if it has a predetermined minimum level of telomerase increasing activity. For example, the extract could be selected if it provides, at a concentration of 25 μg/ml or less, a level of telomerase in keratinocytes or fibroblasts, as measured in a TRAP assay, which is at least 25% greater, and more preferably about 50% greater, than the level observed for a solvent control.

Alternatively, or in addition, the extract, formulated in a suitable solvent medium at one more concentrations, is assayed for wound healing activity in a scratch assay as described above. Preferred extracts for selection include those having wound healing activity, at a concentration of 25 μg/ml or less, which is at least 25% greater, and more preferably at least 50% greater, than that of a solvent control.

Extracts of plant material are prepared accordingly to known methods, such as the extraction methods described in Section IIB above and the exemplary protocol described for Astragalus membranaceus root in Example 1. The plant material is, for example, the root, stem, leaf or flower of a vascular plant, preferably a flowering plant, and more preferably an herb, as defined above. In preferred embodiments, the plant is an Astragalus or Cimicifuga species.

A selected extract can be used to formulate a pharmaceutical or cosmetic composition, comprising a pharmaceutical or cosmetic carrier. Suitable formulations for preparing pharmaceutical or cosmetic compositions of telomerase activating and/or wound healing extracts are described further in Sections V and II, respectively.

### EXAMPLES

The following examples are intended to illustrate but not to limit the invention.

### Example 1

### Exemplary Extraction Protocol

Raw herbal material (Astragalus membranaceus root) was soaked in 95% aqueous EtOH, at a level of 100 mL solvent/20 g plant material, for 30 min, then refluxed for 2 hours. The solid material was filtered, and the reflux step was repeated with fresh solvent. The solvent fractions were combined. This solution could be used directly for screening or dried to obtain the solid extract. Total yields from extraction of 20 g samples of plant material from two sources were, respectively, 1.8 g Mongolia) and 3.2 g (ShanXi).

### Example 2

### Short Term and Long Term Photoaging Assays

Keratinocytes were plated three days prior to (1) addition of vehicle control, (2) addition of 25 μg/ml extract GRN925, or (3) transduction with 10 MOI (multiplicity of infection) AdhTERT. At this point cells were about 40% confluent.

Short term experiment: After 3 additional days (at about 75% confluence), medium was removed, and the cells were rinsed twice with PBS. Cells in each of groups (1), (2) and (3) were then exposed to (a) 7.5 mJ/cm² UVB radiation, (b) 10 mJ/cm²UVB radiation, or (c) ambient radiation. One day later, the cells were harvested, and the extent of apoptosis was determined by an annexin V-FACS assay, using the Immunotech Annexin V-FITC kit. (For descriptions of this assay see e.g. Vermes et al., J. Immunol. Methods 184(1):39-51, 1995; Koopman et al., Blood 84(5): 1415-20, 1994; Wilkins et al. Cytometry 48(1) 14-9, 2002.)

Long term experiment: After 3 additional days (at about 75% confluence), the cells were passaged onto new culture dishes, trypsinized, counted, and replated at a density of 3.5×10⁵ cells/100 cm² in fresh media. The cells were allowed to grow for an additional 5-6 days, to regain about 75% confluence, then treated as above (removal of medium, UVB exposure, and apoptosis assay).

### Example 3

### Exemplary Cosmetic Formulations

The following are examples of cosmetic formulations incorporating the subject extracts. In each case, “Extract” refers to an extract of an Astragalus or Cimicifuga species, preferably an Astragalus extract, and most preferably an Astragalus membranaceus root extract, prepared as described in Example A.

An exemplary oil-in-water emulsion can be prepared using the following ingredients:

| | Ingredient | Weight % |
|---|---|---|
| Phase A | Water U.S.P. | (to 100) |
| | Disodium EDTA | 0.15 |
| | Glycerin | 5 |
| Phase B | Cetyl hydroxy ethyl cellulose | 0.15 |
| | Methyl Paraben | 0.25 |
| Phase C | Cetyl alcohol | 0.5 |
| | Stearyl alcohol | 0.5 |
| | Behenyl alcohol | 0.5 |
| | Cetyl ricinoleate | 3 |
| | Steareth-2 (Brij 72) | 1 |
| | Distearyldimonium chloride | 0.25 |
| | Propyl Paraben | 0.1 |
| | Myristyl myristate | 1.5 |
| | Caprylic/Capritryglycerides | 1.5 |
| | Mineral oil | 2 |
| | Sugar fatty acid ester* | 1 |
| | Polypropylene glycol-15 stearyl ether | 1 |
| Phase D | Dimethicone 10 CST (Dow Corning) | 2 |
| Phase E | Extract | (to give 2.5% w/v) |
| | Isopropanol | 10 |
| | Benzyl alcohol | 10 |
| Phase F | 50% NaOH | to pH 7 |

*For example, a sucrose polyester having a degree of esterification of 7-8, where the fatty acids are C18 mono- and/or di-unsaturated and C22 saturated, e.g. SEFA Cottonate.

The phase A components are blended at a temperature of about 70-80° C., and the phase B components are then added at the same temperature and blended to form a uniform mixture. The phase C components are milled to obtain an acceptably smooth mixture, then added to the above mixture. The mixture is blended and allowed to cool to about about 45° C., at which point dimethicone (phase D) is added, followed by the extract in a lower alcohol such as ethanol (phase E), and NaOH (phase F) to pH 7.

Further exemplary oil-in-water emulsions can be prepared from ingredients as shown in the tables below, again using conventional formulating techniques, such as follows. Distilled water (Phase A) is purged with nitrogen, followed by addition of Phase B ingredients. Phase C ingredients are then dispersed into Phase A/B until uniform, heating to about 75° C. Phase D ingredients are blended separately and heated to about 75° C., then blended into phases A/B/C under nitrogen for approx. 30 minutes, followed by addition of the combined phase E ingredients. The mixture is blended until homogeneous and then cooled. The phase F, G, and H ingredients are added, respectively, to the mixture when it has cooled to 50° C. (phase F), 40° C. (phase G) and 30° C. (phase H). Mixing is continued until the mixture is uniform.

| | Ingredient | Weight % |
|---|---|---|
| Phase B | Glycerin | 5 |
| Phase C | Glycerin | 1 |
| | EDTA | 0.1 |
| | Carbopol 954 | 0.5-0.68 |
| | Carbopol 1382 | 0.1 |
| Phase D | Cetyl alcohol | 0.72 |
| | Stearyl alcohol | 0.48 |
| | Stearic acid | 0.1 |
| | PEG-100 stearate | 0.1 |
| | Arlatone 2121 | 1 |
| | Silicone Q21403 | 2 |
| | Sugar fatty acid ester | 0.67 |
| | Tocopherol acetate | 0-0.5 |
| | Niacinamide | 2 |
| Phase E | Distilled water | 2 |
| | NaOH | (to pH 7) |
| Phase F | Urea | 0-2 |
| | D-Panthenol | 0-0.5 |
| | Distilled water | 5 |
| Phase G | Glydant Plus | 0.1 |
| | Glycerin | 1 |
| | Distilled water | 1 |
| Phase H | Methyl/isopropyl isostearate | 1.3-2.6 |
| | BHT | 0.05 |
| | Tween 20 | 0.04 |
| | Extract | (to give 5% w/v) |
| Phase B | Glycerin | 6 |
| Phase C | Glycerin | 3 |
| | Carbopol 954 | 0.4 |
| | EDTA | 0.1 |
| Phase D | Cetyl palmitate | 1.5 |
| | Cetyl alcohol | 2.25 |
| | Stearyl alcohol | 1.5 |
| | Stearic acid | 0.31 |
| | PEG-100 stearate | 0.31 |
| | Silicone wax DC2501 | 2 |
| | DC 3225C | 1.88 |
| | Dimethicone 200/350 CST | 0.63 |
| | Tocopherol acetate | 0-0.5 |
| | Niacinamide | 2 |
| Phase E | Distilled water | 2 |
| | NaOH | (to pH 7) |
| Phase F | D-Panthenol | 0-0.5 |
| | Distilled water | 0-5 |
| Phase G | Glydant Plus | 0.1 |
| | Distilled water | 1 |
| | Glycerin | 1 |
| Phase H | Isopropyl palmitate | 1.25 |
| | Tween 80 | 0-0.04 |
| | BHT | 0-0.05 |
| | Extract | (to give 2.5% w/v) |

A high internal phase water-in-oil emulsion can be prepared using the following components:

| Ingredient | Weight % |
|---|---|
| Extract | (to give 1.0% w/v) |
| 1,3-dimethyl-2-imidazolidinone | 0.2 |
| Brij 92 (Polyoxyethylene (2) oleyl ether) | 5 |
| Bentone 38 (Quaternium 18-hectorite) | 0.5 |
| MgSO₄•7H₂O | 0.3 |
| Butylated hydroxy toluene | 0.01 |
| Perfume | qs |
| Water | (to 100) |

An oil-in-water cream can be prepared using the following components:

| Ingredient | Weight % |
|---|---|
| Extract | (to give 2.5% w/v) |
| Glycolic Acid | 8 |
| Mineral oil | 4 |
| 1,3-dimethyl-2-imidazolidinone | 1 |
| Brij 56 (Cetyl alcohol POE (10)) | 4 |
| Cetyl alcohol | 4 |
| Triethanolamine | 0.75 |
| Butane-1,3-diol | 3 |
| Xanthan gum | 0.3 |
| Perfume | qs |
| Butylated hydroxy toluene | 0.01 |
| Water | (to 100) |

An alcoholic lotion can be prepared using the following components:

| Ingredient | Weight % |
|---|---|
| Extract | (to give 6.5% w/v) |
| 1,3-dimethyl-2-imidazolidinone | 0.1 |
| Ethanol | 40 |
| Perfume | qs |
| Butylated hydroxy toluene | 0.01 |
| Water | (to 100) |

A sunscreen cream can be prepared using the following components:

| Ingredient | Weight % |
|---|---|
| Extract | (to give 4.0% w/v) |
| 1,3-dimethyl-2-imidazolidinone | 0.2 |
| Silicone oil 200 cts | 7.5 |
| Glycerylmonostearate | 3 |
| Cetosteryl alcohol | 1.6 |
| Polyoxyethylene-(20)-cetyl alcohol | 1.4 |
| Xanthan gum | 0.5 |
| Parsol 1789 (Butylmethoxydibenzoylmethane) | 1.5 |
| Parsol MCX (Octyl methoxycinnamate) | 7 |
| Perfume | qs |
| Color | qs |
| Water | (to 100) |

A non-aqueous skin care composition can be prepared using the following components:

| Ingredient | Weight % |
|---|---|
| Extract | (to give 7.0% w/v) |
| 1,3-dimethyl-2-imidazolidinone | 1 |
| Silicone gum SE-30^{a} | 10 |
| Silicone fluid 345^{b} | 20 |
| Silicone fluid 344^{c} | (to 100) |
| Squalene | 10 |
| Linoleic acid | 0.01 |
| Cholesterol | 0.03 |
| 2-hydroxy-n-octanoic acid | 0.7 |
| Vitamin E linoleate | 0.5 |
| Herbal oil | 0.5 |
| Ethanol | 2 |

^{a}Dimethyl silicone polymer, MW ≧ 50,000, available from GEC
^{b}Dimethyl siloxane cyclic pentamer, available from Dow Corning Corp.
^{c}Dimethyl siloxane tetramer, available from Dow Corning Corp.

1-11. (canceled)

12. A method of increasing telomerase activity in a cell or tissue, comprising
contacting said cell or tissue with a composition comprising an extract of an Astragalus or Cimicifuga species,
wherein the extract has telomerase inducing activity, such that, when said extract is formulated in a solvent at a concentration of 25 μg/ml or less, it is able to produce a level of telomerase activity in a keratinocyte or fibroblast cell, as measured in a Telomere Repeat Amplification Protocol assay, which is at least 25% greater than that measured for said solvent alone.

13. The method of claim 12, wherein the species is an Astragalus species.

14. The method of claim 13, wherein the species is selected from the group consisting of A. membranaceus, A. trojanus, A. zahlbruckneri, A. brachypterus, A. microcephalus, A. peregrinus, A. caprinus, A. melanophrurius, A. oleifolius, A. trigonus, A. spinosus, and A. verrucosus.

15. The method of claim 14, wherein the species is Astragalus membranaceus.

16. The method of claim 12, wherein the species is a Cimicifuga species selected from the group consisting of C. racemosa, C. dahurica, C. foetida, and C. acerina.

17. The method of claim 15, wherein the extract is from the root of the plant.

18. The method of claim 12, further comprising the step of, prior to said contacting, identifying a cell or tissue in which an increase in telomerase activity is desired.

19-24. (canceled)

25. The method of claim 12, wherein the concentration of said extract in said composition is from 0.1 to 50% (w/v).

26. The method of claim 25, wherein said concentration is from 0.25 to 25% (w/v).

27-54. (canceled)

55. The method of claim 15, wherein the extract is obtained by extracting Astragalus membranaceus root with a polar solvent selected from the group consisting of water, lower alcohols, and combinations thereof.

56. The method of claim 55, wherein the extract is obtained by a process that comprises:
(a) heating a mixture containing Astragalus membranaceus root in a solvent containing at least 75% ethanol and the remainder water;
(b) filtering the solid from the resulting mixture;
(c) repeating steps (a)-(b) with the solid of step (b) and fresh solvent; and
(d) combining the filtrates from steps (b).
